(19) United States
(12) Patent Application Publication
(10) Pub. No.: US 2025/0040673 A1
(43) Pub. Date: Feb. 6, 2025

(54) PACKAGING SOLUTIONS

(71) Applicant: Bausch + Lomb Ireland Limited, Dublin (IE)

(72) Inventors: Mark Mis, Rush, NY (US); Jade Russell, Perry, NY (US); Alok Kumar Awasthi, Pittsford, NY (US)

(21) Appl. No.: 18/774,016

(22) Filed: Jul. 16, 2024

Related U.S. Application Data
(60) Provisional application No. 63/529,551, filed on Jul. 28, 2023.

(51) Int. Cl. A45C 11/00; B65B 25/00; B65B 55/02; B65B 55/22

(52) U.S. Cl. CPC A45C 11/005; B65B 25/008; B65B 55/02; B65B 55/22

(57) ABSTRACT

A packaging system for the storage of an unused ophthalmic device is disclosed. The packaging system includes a sealed container containing one or more unused ophthalmic devices immersed in an aqueous packaging solution that includes hyaluronic acid or a salt or a derivative thereof and tremella. The aqueous packaging solution has an osmolality of at least about 150 mOsm/kg, a pH of about 6 to about 9 and is sterilized.

### PRIORITY CLAIM

The present application claims priority to U.S. Provisional Patent Application Ser. No. 63/529,551, entitled “Packaging Solutions,” filed Jul. 28, 2023, the content of which is incorporated by reference herein in its entirety.

### BACKGROUND

Blister-packs and glass vials are typically used to individually package each soft contact lens for sale to a customer. Saline or deionized water is commonly used to store the lens in the blister-packs. Because lens material may tend to stick to itself and to the lens package, packaging solutions for blister-packs have sometimes been formulated with various components to reduce or eliminate lens folding and sticking.

### SUMMARY

In accordance with an illustrative embodiment, a packaging system for the storage of an unused ophthalmic device comprises a sealed container containing one or more unused ophthalmic devices immersed in an aqueous packaging solution comprising (a) hyaluronic acid or a salt or a derivative thereof and (b) tremella, wherein the aqueous packaging solution has an osmolality of at least about 150 mOsm/kg, a pH of about 6 to about 9 and is sterilized.

In accordance with yet another illustrative embodiment, a method of preparing a packaging system comprising a storable, sterile ophthalmic device comprises:
(a) providing an unused ophthalmic device;
(b) immersing the unused ophthalmic device in an aqueous packaging solution comprising (i) hyaluronic acid or a salt or a derivative thereof and (ii) tremella, wherein the aqueous packaging solution has an osmolality of at least about 150 mOsm/kg and a pH in the range of about 6 to about 9;
(c) packaging the aqueous packaging solution and the unused ophthalmic device in a manner preventing contamination of the unused ophthalmic device by microorganisms; and
(d) sterilizing the packaged solution and unused ophthalmic device.

### DETAILED DESCRIPTION

The illustrative embodiments described herein are directed to packaging systems for the storage of ophthalmic devices intended for direct contact with body tissue or body fluid, e.g., direct contact in the eye. It is highly desirable that an ophthalmic device such as a contact lens be as comfortable as possible for wearers. Manufacturers of contact lenses are continually working to improve the comfort of the lenses. Nevertheless, many people who wear contact lenses still experience dryness or eye irritation throughout the day and particularly towards the end of the day. An insufficiently wetted lens at any point in time will cause significant discomfort to the lens wearer. Although wetting drops can be used as needed to alleviate such discomfort, it would certainly be desirable if such discomfort did not arise in the first place.

Hyaluronic acid is a linear polysaccharide (long-chain biological polymer) formed by repeating disaccharide units consisting of D-glucuronic acid and N-acetyl-D-glucosamine linked by B (1-3) and B (1-4) glycosidic linkages. Hyaluronic acid is a glycosaminoglycan, and is distinguished from the other glycosaminoglycans, as it is free from covalent links to protein and sulphonic groups. Hyaluronic acid is ubiquitous in animals with the highest concentration found in soft connective tissue. It plays an important role for both mechanical and transport purposes in the body, e.g., it gives elasticity to the joints and rigidity to the vertebrate disks, and it is also an important component of the vitreous body of the eye.

Hyaluronic acid, because of its high degree of hydration, is likely responsible for increasing the resistance of biological tissues or cells to compression. Also, the viscoelastic properties of hyaluronic acid, that is, hard elastic under static conditions though less viscous under small shear forces enables hyaluronic acid to basically function as a shock absorber for cells and tissues. The hyaluronic acid properties are dependent on the molecular weight, the solution concentration, and physiological pH. At low concentrations the individual chains entangle and form a continuous network in solution, which gives the system interesting properties such as pronounced viscoelasticity and pseudoplasticity that is unique for a water-soluble polymer at low concentration.

As discussed above, blister-packs are typically used to individually package each soft contact lens for sale to a customer. When sealing the blister-packs with the contact lens immersed in the packaging solution, the sealed blister packs are typically subjected to two sterilization procedures involving autoclaving. However, during the autoclaving procedures, the hyaluronic acid degrades thereby becoming less effective.

Accordingly, these problems are overcome by the illustrative embodiments disclosed herein which provide an improved packaging system for ophthalmic devices such as a contact lens such that the lens would be more lubricious and comfortable to wear in actual use thereby allowing for extended wear of the lens without irritation or other adverse effects to the cornea. In non-limiting illustrative embodiments, a packaging system for ophthalmic devices as disclosed herein includes an aqueous packaging solution containing at least hyaluronic acid or a salt thereof and tremella such that the hyaluronic acid or a salt or derivative thereof degrades at a slower rate after autoclaving as compared to a packaging system including an aqueous packaging solution containing hyaluronic acid or a salt or derivative thereof alone. The ophthalmic device will therefore be more comfortable to wear in actual use and allow for extended wear of the lens without adverse effects to the cornea. Hydrophilic and/or lubricious surfaces of the ophthalmic devices herein such as contact lenses substantially prevent or limit the adsorption of tear lipids and proteins on, and their eventual absorption into, the lenses, thus preserving the clarity of the contact lenses. This, in turn, preserves their performance quality thereby providing a higher level of comfort to the wearer.

As used herein, the term “ophthalmic device” refers to devices that reside in or on the eye. These lenses can provide optical correction, wound care, drug delivery, diagnostic functionality or cosmetic enhancement or effect or a combination of these properties. Representative examples of such devices include, but are not limited to, soft contact lenses, e.g., a soft, hydrogel lens; soft, non-hydrogel lens and the like, hard contact lenses, e.g., a hard, gas permeable lens material and the like, intraocular lenses, overlay lenses, ocular inserts, optical inserts and the like. As is understood by one skilled in the art, a lens is considered to be “soft” if it can be folded back upon itself without breaking. Any material known to produce an ophthalmic device including a contact lens can be used herein.

The ophthalmic devices for use in the packaging systems disclosed herein can be any material known in the art capable of forming an ophthalmic device as described above. In an embodiment, an ophthalmic device includes devices which are formed from material not hydrophilic per se. Such devices are formed from materials known in the art and include, by way of example, polysiloxanes, perfluoropolyethers, fluorinated poly (meth) acrylates or equivalent fluorinated polymers derived, e.g., from other polymerizable carboxylic acids, polyalkyl (meth) acrylates or equivalent alkylester polymers derived from other polymerizable carboxylic acids, or fluorinated polyolefins, such as fluorinated ethylene propylene polymers, or tetrafluoroethylene, preferably in combination with a dioxol, e.g., perfluoro-2,2-dimethyl-1,3-dioxol. Representative examples of suitable bulk materials include, but are not limited to, lotrafilcon A, neofocon, pasifocon, telefocon, silafocon, fluorsilfocon, paflufocon, silafocon, elastofilcon, fluorofocon or Teflon® AF materials, such as Teflon® AF 1600 or Teflon® AF 2400 which are copolymers of about 63 to about 73 mol % of perfluoro-2,2-dimethyl-1,3-dioxol and about 37 to about 27 mol % of tetrafluoroethylene, or of about 80 to about 90 mol % of perfluoro-2,2-dimethyl-1,3-dioxol and about 20 to about 10 mol % of tetrafluoroethylene.

In another embodiment, an ophthalmic device includes a device which is formed from material hydrophilic per se, since reactive groups, e.g., carboxy, carbamoyl, sulfate, sulfonate, phosphate, amine, ammonium or hydroxy groups, are inherently present in the material and therefore also at the surface of an ophthalmic device manufactured therefrom. Such devices are formed from materials known in the art and include, by way of example, polyhydroxyethyl acrylate, polyhydroxyethyl methacrylate (HEMA), polyvinyl pyrrolidone (PVP), polyacrylic acid, polymethacrylic acid, polyacrylamide, polydimethylacrylamide (DMA), polyvinyl alcohol and the like and copolymers thereof, e.g., from two or more monomers selected from hydroxyethyl acrylate, hydroxyethyl methacrylate, N-vinyl pyrrolidone, acrylic acid, methacrylic acid, acrylamide, dimethyl acrylamide, vinyl alcohol and the like. Representative examples of suitable bulk materials include, but are not limited to, polymacon, tefilcon, methafilcon, deltafilcon, bufilcon, phemfilcon, ocufilcon, focofilcon, etafilcon, hefilcon, vifilcon, tetrafilcon, perfilcon, droxifilcon, dimefilcon, isofilcon, mafilcon, nelfilcon, atlafilcon and the like. Examples of other suitable bulk materials include balafilcon A, hilafilcon A, alphafilcon A, bilafilcon B and the like.

In another embodiment, an ophthalmic device includes a device which is formed from materials which are amphiphilic segmented copolymers containing at least one hydrophobic segment and at least one hydrophilic segment which are linked through a bond or a bridge member.

It is particularly useful to employ biocompatible materials herein including both soft and rigid materials commonly used for ophthalmic lenses, including contact lenses. In general, non-hydrogel materials or rigid materials are hydrophobic polymeric materials that do not contain water in their equilibrium state. Typical non-hydrogel materials and rigid materials comprise silicone acrylics, such as those formed from a bulky silicone monomer (e.g., tris (trimethylsiloxy) silylpropyl methacrylate, commonly known as “TRIS” monomer), methacrylate end-capped poly (dimethylsiloxane) prepolymer, or silicones having fluoroalkyl side groups (polysiloxanes are also commonly known as silicone polymers).

In another embodiment, an ophthalmic device includes a device which is a hydrogel. Hydrogels in general are a well-known class of materials that comprise hydrated, crosslinked polymeric systems containing water in an equilibrium state. Accordingly, hydrogels are copolymers prepared from hydrophilic monomers. In the case of silicone hydrogels, the hydrogel copolymers are generally prepared by polymerizing a monomeric mixture containing at least one ophthalmic device-forming silicone-containing monomer and at least one ophthalmic device-forming hydrophilic monomer. Either the silicone-containing monomer or the hydrophilic monomer can function as a crosslinking agent (a crosslinker being defined as a monomer having multiple polymerizable functionalities) or a separate crosslinker may be employed. Silicone hydrogels typically have a water content between about 10 to about 80 weight percent.

In some embodiments, an ophthalmic device can be obtained from a polymerization product of a monomeric mixture comprising at least one ophthalmic device-forming hydrophilic monomer.

Suitable one or more hydrophilic comonomers include, for example, unsaturated carboxylic acids, acrylamides, vinyl lactams, hydroxyl-containing-(meth) acrylates, hydrophilic vinyl carbonates, hydrophilic vinyl carbamates, hydrophilic oxazolones, and poly (alkene glycols) functionalized with polymerizable groups and the like and mixtures thereof.

As used herein, the term “(meth)” denotes an optional methyl substituent. Thus, terms such as “(meth) acrylate” denotes either methacrylate or acrylate, and “(meth) acrylamide” denotes either methacrylamide or acrylamide.

Representative examples of unsaturated carboxylic acids include, but are not limited to, methacrylic acid, acrylic acid and the like and mixtures thereof. Representative examples of acrylamides include, but are not limited to, alkylamides such as N,N-dimethylacrylamide, N,N-dimethylmethacrylamide and the like and mixtures thereof. Representative examples of cyclic lactams include, but are not limited to, N-vinyl-2-pyrrolidone, N-vinyl caprolactam, N-vinyl-2-piperidone and the like and mixtures thereof. Representative examples of hydroxyl-containing (meth) acrylates include, but are not limited to, 2-hydroxyethyl methacrylate (HEMA), glycerol methacrylate and the like and mixtures thereof. Additional device-forming hydrophilic comonomers include, for example, the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable device-forming hydrophilic comonomers will be apparent to one skilled in the art. Mixtures of the foregoing device-forming hydrophilic comonomers can also be used in the monomeric mixtures herein.

In accordance with one or more additional non-limiting illustrative embodiments, as may be combined with one or more of the preceding paragraphs, the one or more hydrophilic comonomers can be present in the monomeric mixture in an amount ranging from about 20 wt. % to about 80 wt. %, based on the total weight of the monomeric mixture. In another illustrative embodiment, the one or more hydrophilic comonomers can be present in the monomeric mixture in an amount ranging from about 30 wt. % to about 60 wt. %, based on the total weight of the monomeric mixture.

The monomer mixtures may also include a second ophthalmic device-forming comonomer including a copolymerizable group and a reactive functional group. The copolymerizable group is preferably an ethylenically unsaturated group, such that this device-forming monomer copolymerizes with the hydrophilic ophthalmic device-forming monomer and any other device-forming monomers in the initial device-forming monomer mixture. Additionally, the other ophthalmic device-forming comonomer can include a reactive functional group that reacts with a complementary reactive group of the copolymer such as the reaction product of one or more polymerizable polyhydric alcohols and one or more polymerizable fluorine-containing monomers. In other words, after the ophthalmic device is formed by copolymerizing the monomeric mixture, the reactive functional groups provided by the second device-forming monomers remain to react with a complementary reactive moiety of the copolymer.

In an embodiment, reactive groups of the second ophthalmic device-forming comonomers include epoxide groups. Accordingly, the second ophthalmic device-forming comonomers are those that include both an ethylenically unsaturated group (that permits the monomer to copolymerize with the hydrophilic ophthalmic device-forming monomer) and the epoxide group (that does not react with the hydrophilic ophthalmic device-forming monomer but remains to react with a copolymer, e.g., the reaction product of one or more polymerizable polyhydric alcohols and one or more polymerizable fluorine-containing monomers). Suitable second ophthalmic device-forming monomers include, for example, glycidyl methacrylate, glycidyl acrylate, glycidyl vinylcarbonate, glycidyl vinylcarbamate, and 4-vinyl-1-cyclohexene-1,2-epoxide.

In accordance with one or more additional non-limiting illustrative embodiments, as may be combined with one or more of the preceding paragraphs, the one or more second ophthalmic device-forming comonomers can be one or more ophthalmic device-forming silicone comonomers. Representative ophthalmic device-forming silicone comonomers for use in the formation of, for example, silicone hydrogels, are well known in the art and numerous examples are provided in U.S. Pat. Nos. 4,136,250; 4,153,641; 4,740,533; 5,034,461; 5,070,215; 5,260,000; 5,310,779; and 5,358,995. Specific examples of suitable materials for use herein include those disclosed in U.S. Pat. Nos. 5,310,779; 5,387,662; 5,449,729; 5,512,205; 5,610,252; 5,616,757; 5,708,094; 5,710,302; 5,714,557 and 5,908,906, the contents of which are incorporated by reference herein.

In an illustrative embodiment, as may be combined with one or more of the preceding paragraphs, the ophthalmic device-forming silicone comonomers can include, as a class of representative ophthalmic device-forming silicone comonomers, one or more non-bulky organosilicon-containing monomers. An “organosilicon-containing monomer” as used herein contains at least one [siloxanyl] or at least one [silyl-alkyl-siloxanyl] repeating unit, in a monomer, macromer or prepolymer. In an illustrative embodiment, an example of a non-bulky organosilicon-containing monomers is represented by a structure of Formula Ia:

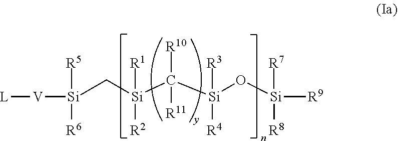

wherein L is ethylenically unsaturated polymerizable group, V is a linking group or a bond; R¹, R², R³, R⁴, R⁵, R⁶, R⁷, R⁸, and R⁹ are independently hydrogen, an alkyl group, a haloalkyl group, a cycloalkyl group, a heterocycloalkyl group, an alkenyl group, a halo alkenyl group, or an aryl group; R¹⁰ and R¹¹ are independently hydrogen or an alkyl group wherein at least one of R¹⁰ and R¹¹ is hydrogen; y is 2 to 7 and n is 1 to 100 or from 1 to 20.

Ethylenically unsaturated polymerizable groups are well known to those skilled in the art. Suitable ethylenically unsaturated polymerizable groups include, for example, (meth) acrylates, vinyl carbonates, O-vinyl carbamates, N-vinyl carbamates, and (meth) acrylamides.

Linking groups can be any divalent radical or moiety and include, for example, a substituted or unsubstituted C₁ to C₁₂ alkyl group, an alkyl ether group, an alkenyl group, an alkenyl ether group, a halo alkyl group, a substituted or unsubstituted siloxane group, and monomers capable of propagating ring opening.

In one embodiment, V is a (meth) acrylate, L is a C₁ to C₁₂ alkylene group, R¹, R², R³, R⁴, R⁵, R⁶, R⁷, R⁸, and R⁹ are independently a C₁ to C₁₂ alkyl group, R¹⁰ and R¹¹ are independently H or a C₁ to C₁₂ alkyl group, y is 2 to 7 and n is 3 to 8.

In one embodiment, V is a (meth) acrylate, L is a C₁ to C₆ alkyl group, R¹, R², R³, R⁴, R⁵, R⁶, R⁷, R⁸, and R⁹ are independently a C₁ to C₆ alkyl group, R¹⁰ and R¹¹ are independently H or a C₁ to C₆ alkyl group, y is 2 to 7 and n is 1 to 20.

Non-bulky organosilicon-containing monomers represented by a structure of Formula Ia are known in the art, see, e.g., U.S. Pat. Nos. 7,915,323, 7,994,356, 8,420,711, 8,827,447 and 9,039,174, the contents of which are incorporated by reference herein.

In an illustrative embodiment, as may be combined with one or more of the preceding paragraphs, the one or more non-bulky organosilicon-containing monomers can also comprise a compound represented by a structure of Formula Ib:

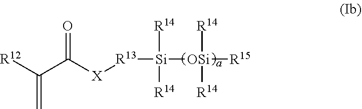

wherein R¹² is H or methyl; X is O or NR¹⁶; wherein R¹⁶ is selected from H, or C₁ to C₄ alkyl, which may be further substituted with one or more hydroxyl groups, and in some embodiments is H or methyl; R¹³ is a divalent alkyl group, which may further be functionalized with a group selected from the group consisting of ether groups, hydroxyl groups, carbamate groups and combinations thereof, and in another embodiment a C₁ to C₆ alkylene group which may be substituted with ether, hydroxyl and combinations thereof, and in yet another embodiment a C₁ or C₃ to C₄ alkylene group which may be substituted with ether, hydroxyl and combinations thereof; each R¹⁴ is independently a phenyl or a C₁ to C₄ alkyl group which may be substituted with fluorine, hydroxyl or ether, and in another embodiment each R¹⁴ is independently selected from ethyl and methyl groups, and in yet another embodiment, each R¹⁴ is methyl; R¹⁵ is a C₁ to C₄ alkyl group; a is 2 to 50, and in some embodiments 5 to 15.

Non-bulky organosilicon-containing monomers represented by a structure of Formula Ib are known in the art, see, e.g., U.S. Pat. Nos. 8,703,891, 8,937,110, 8,937,111, 9,156,934 and 9,244,197, the contents of which are incorporated by reference herein.

Representative examples of the non-bulky organosilicon-containing monomers include:
M1EDS6: a compound having the structure and available from Gelest:

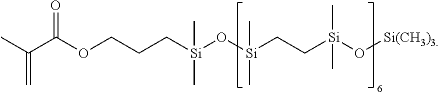

MCR-M11: a compound having the structure:

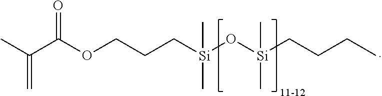

M1-MCR-C12: a compound having the structure:

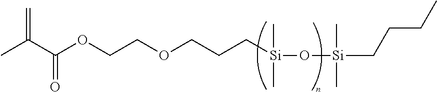

wherein n is an average of 12.

In an illustrative embodiment, as may be combined with one or more of the preceding paragraphs, the one or more ophthalmic device-forming silicone comonomers can include, as a class of representative ophthalmic device-forming silicone comonomers, one or more bulky silicone-containing monomers. Suitable one or more bulky silicone monomers include, for example, one or more bulky siloxane monomers such as polysiloxanylalkyl (meth) acrylic monomers, bulky polysiloxanylalkyl carbamate monomers and mixtures thereof. In one embodiment, a representative example of a bulky silicone-containing monomer is represented by a structure of Formula II:

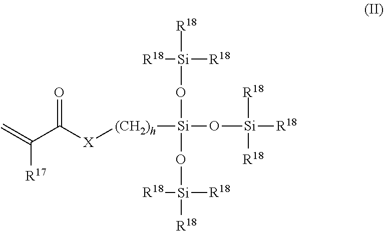

wherein X denotes —O— or —NR¹⁹—, where each R¹⁹ is hydrogen or a C₁-C₄ alkyl group; R¹⁷ independently denotes hydrogen or methyl; each R¹⁸ independently denotes a lower alkyl radical such as a C₁-C₆ group, a phenyl radical or a group represented by the following structure:

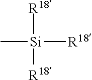

wherein each R^{18′} independently denotes a lower alkyl radical or a phenyl radical; and h is 1 to 10; or is represented by a structure of Formula III:

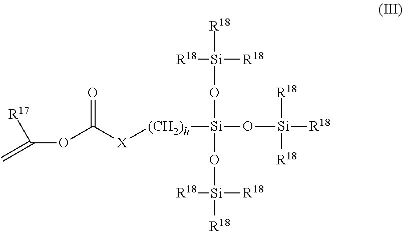

wherein X denotes —NR¹⁹— wherein R¹⁹ denotes hydrogen or a C₁-C₄ alkyl; R¹⁷ denotes hydrogen or methyl; each R¹⁸ independently denotes a lower alkyl radical, a phenyl radical or a group represented by the following structure:

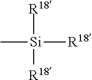

wherein each R¹⁸′ independently denotes a lower alkyl radical or a phenyl radical; and h is 1 to 10.

Representative examples of bulky silicone-containing monomers include 3-methacryloyloxypropyltris (trimethylsiloxy) silane or tris (trimethylsiloxy) silylpropyl methacrylate, sometimes referred to as TRIS and tris (trimethylsiloxy) silylpropyl vinyl carbamate, sometimes referred to as TRIS-VC, pentamethyldisiloxanyl methylmethacrylate, phenyltetramethyl-disiloxanylethyl acetate, and methyldi (trimethylsiloxy) methacryloxymethyl silane, (3-methacryloxy-2-hydroxy propoxy) propyl bis (trimethyl siloxy) methyl silane, sometimes referred to as Sigma and the like and mixtures thereof. In one embodiment, the bulky silicone-containing monomer is a tris (trialkylsiloxy) silylalkyl methacrylate-containing monomer such as a tris (trimethylsiloxy) silylpropyl methacrylate-containing monomer.

Such bulky monomers may be copolymerized with a silicone macromonomer, which is a poly (organosiloxane) capped with an unsaturated group at two or more ends of the molecule. U.S. Pat. No. 4,153,641 discloses, for example, various unsaturated groups such as acryloxy or methacryloxy groups.

In an illustrative embodiment, as may be combined with one or more of the preceding paragraphs, the one or more ophthalmic device-forming silicone comonomers can include, as a class of representative ophthalmic device-forming silicone comonomers, one or more silicone-containing vinyl carbonate or vinyl carbamate monomers. Suitable one or more silicone-containing vinyl carbonate or vinyl carbamate monomers include, for example, 1,3-bis [4-vinyloxycarbonyloxy) but-1-yl] tetramethyl-disiloxane; 3-(trimethylsilyl) propyl vinyl carbonate; 3-(vinyloxycarbonylthio) propyl-[tris (trimethylsiloxy) silane]; 3-[tris (trimethylsiloxy) silyl] propyl vinyl carbamate; 3-[tris (trimethylsiloxy) silyl] propyl allyl carbamate; 3-[tris (trimethylsiloxy) silyl] propyl vinyl carbonate; t-butyldimethylsiloxyethyl vinyl carbonate; trimethylsilylethyl vinyl carbonate; trimethylsilylmethyl vinyl carbonate and the like and mixtures thereof.

In an illustrative embodiment, as may be combined with one or more of the preceding paragraphs, the one or more ophthalmic device-forming silicone comonomers can include, as a class of representative ophthalmic device-forming silicone comonomers, one or more polyurethane-polysiloxane macromonomers (also sometimes referred to as prepolymers), which may have hard-soft-hard blocks like traditional urethane elastomers. They may be end-capped with a hydrophilic monomer such as HEMA. Examples of such silicone urethanes are disclosed in a variety or publications, including Lai, Yu-Chin, “The Role of Bulky Polysiloxanylalkyl Methacryates in Polyurethane-Polysiloxane Hydrogels,” Journal of Applied Polymer Science, Vol. 60, 1193-1199 (1996). PCT Published Application No. WO 96/31792 discloses examples of such monomers, which disclosure is hereby incorporated by reference in its entirety. Further examples of silicone urethane monomers are represented by Formulae IV and V:

E(*D*A*D*G)ₐ*D*A*D*E′; or (IV)

E(*D*G*D*A)ₐ*D*A*D*E′; or (V)

wherein:
D independently denotes an alkyl diradical, an alkyl cycloalkyl diradical, a cycloalkyl diradical, an aryl diradical or an alkylaryl diradical having 6 to about 30 carbon atoms;
G independently denotes an alkyl diradical, a cycloalkyl diradical, an alkyl cycloalkyl diradical, an aryl diradical or an alkylaryl diradical having 1 to about 40 carbon atoms and which may contain ether, thio or amine linkages in the main chain;
* denotes a urethane or ureido linkage;
a is at least 1;

A independently denotes a divalent polymeric radical of Formula VI:

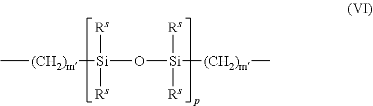

wherein each R⁵ independently denotes an alkyl or fluoro-substituted alkyl group having 1 to about 10 carbon atoms which may contain ether linkages between the carbon atoms; m′ is at least 1; and p is a number that provides a moiety weight of about 400 to about 10,000;

each of E and E′ independently denotes a polymerizable unsaturated organic radical represented by Formula VII:

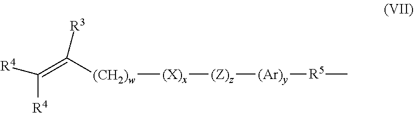

wherein: R³ is hydrogen or methyl;
R⁴ is hydrogen, an alkyl radical having 1 to 6 carbon atoms, or a —CO—Y—R⁶ radical wherein
Y is —O—, —S— or —NH—;
R⁵ is a divalent alkylene radical having 1 to about 10 carbon atoms;
R⁶ is a alkyl radical having 1 to about 12 carbon atoms;
X denotes —CO— or —OCO—;
Z denotes —O— or —NH—;
Ar denotes an aromatic radical having about 6 to about 30 carbon atoms;
w is 0 to 6; x is 0 or 1; y is 0 or 1; and z is 0 or 1.

In an illustrative embodiment, as may be combined with one or more of the preceding paragraphs, the one or more ophthalmic device-forming silicone comonomers can include, as a class of representative ophthalmic device-forming silicone comonomers, one or more silicone-containing urethane monomers represented by Formula VIII:

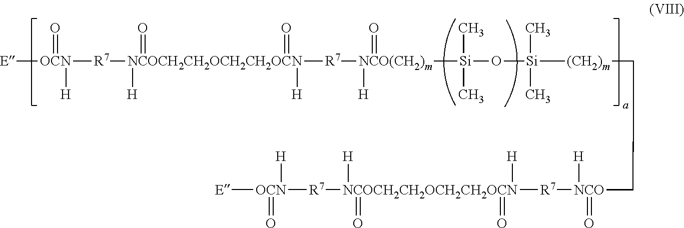

wherein m is at least 1 and is preferably 3 or 4, a is at least 1 and preferably is 1, p is a number which provides a moiety weight of about 400 to about 10,000 and is preferably at least about 30, R⁷ is a diradical of a diisocyanate after removal of the isocyanate group, such as the diradical of isophorone diisocyanate, and each E″ is a group represented by:

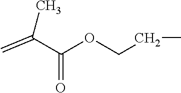

In another embodiment, a silicone hydrogel material comprises (in bulk, that is, in the monomer mixture that is copolymerized) about 5 to about 50 percent, or from about 10 to about 25 percent, by weight of one or more silicone macromonomers, about 5 to about 75 percent, or about 30 to about 60 percent, by weight of one or more polysiloxanylalkyl (meth) acrylic monomers, and about 10 to about 50 percent, or about 20 to about 40 percent, by weight of a hydrophilic monomer. In general, the silicone macromonomer is a poly (organosiloxane) capped with an unsaturated group at two or more ends of the molecule. In addition to the end groups in the above structural formulas, U.S. Pat. No. 4,153,641 discloses additional unsaturated groups, including acryloxy or methacryloxy. Fumarate-containing materials such as those disclosed in U.S. Pat. Nos. 5,310,779; 5,449,729 and 5,512,205 are also useful substrates in accordance with the non-limiting embodiments described herein. The silane macromonomer may be a silicone-containing vinyl carbonate or vinyl carbamate or a polyurethane-polysiloxane having one or more hard-soft-hard blocks and end-capped with a hydrophilic monomer.

In an illustrative embodiment, as may be combined with one or more of the preceding paragraphs, the one or more ophthalmic device-forming silicone comonomers can include, as a class of representative ophthalmic device-forming silicone comonomers, one or more monomers of Formula IX:

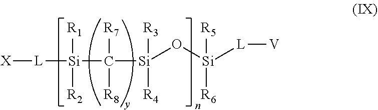

wherein X is the residue of a ring opening agent; L is the same or different and is a linking group or a bond; V is an ethylenically unsaturated polymerizable group; R₁, R₂, R₃, R₄, R₅, and R⁶ are independently hydrogen, an alkyl group, a haloalkyl group, a cycloalkyl group, a heterocycloalkyl group, an alkenyl group, a halo alkenyl group, or an aromatic group; R₇ and R₈ are independently hydrogen or an alkyl group wherein at least one of R₇ or R₈ is hydrogen; y is 2-7 and n is 1-100.

Ring opening agents are well known in the literature. Non-limiting examples of anionic ring opening agents include alkyl lithium, an alkoxide, trialkylsiloxylithium wherein the alkyl group may or may not contain halo atoms.

Linking groups can be any divalent radical or moiety and include substituted or unsubstituted alkyl, alkyl ether, alkenyls, alkenyl ethers, halo alkyls, substituted or unsubstituted siloxanes, and monomers capable of propagating ring opening.

Ethylenically unsaturated polymerizable groups are well known to those skilled in the art. Non-limiting examples of ethylenically unsaturated polymerizable groups would include acrylates, methacrylates, vinyl carbonates, O-vinyl carbamates, N-vinyl carbamates, acrylamides and methacrylamides.

In an illustrative embodiment, as may be combined with one or more of the preceding paragraphs, the one or more ophthalmic device-forming silicone comonomers can include, as a class of representative ophthalmic device-forming silicone comonomers, one or more monomers of Formula X:

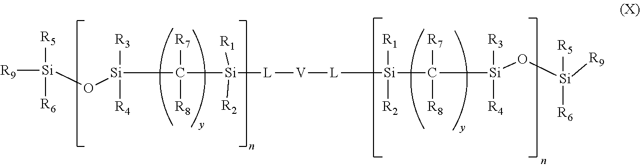

wherein L is the same or different and is a linking group or a bond; V is the same or different and is an ethylenically unsaturated polymerizable group; R₁, R₂, R₃, R₄, R₅, R₆ and R₉ are independently hydrogen, an alkyl group, a haloalkyl group, a cycloalkyl group, a heterocycloalkyl group, an alkenyl group, a halo alkenyl group, or an aromatic group; R₇ and R₈ are independently hydrogen or an alkyl group wherein at least one of R₇ or R₈ is hydrogen; y is 2-7 and n is 1-100.

In an illustrative embodiment, as may be combined with one or more of the preceding paragraphs, the one or more ophthalmic device-forming silicone comonomers can include, as a class of representative ophthalmic device-forming silicone comonomers, one or more monomers of Formulae XI and XII:

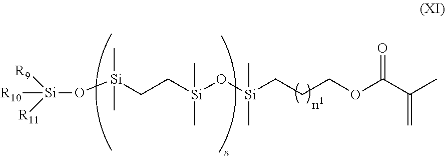

wherein R₉, R₁₀ and R₁₁ are independently hydrogen, an alkyl group, a haloalkyl group or other substituted alkyl groups; n is as defined above and n¹ is 0-10; and,

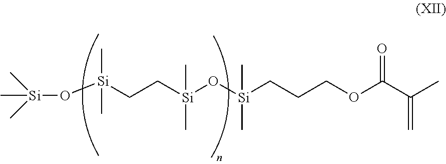

wherein n is 1 to 100, or n is 2 to 80, or n is 3 to 20, or n is 5 to 15.

In an illustrative embodiment, as may be combined with one or more of the preceding paragraphs, the one or more ophthalmic device-forming silicone comonomers can include, as a class of representative ophthalmic device-forming silicone comonomers, one or more monomers of Formulas XIII-XVII:

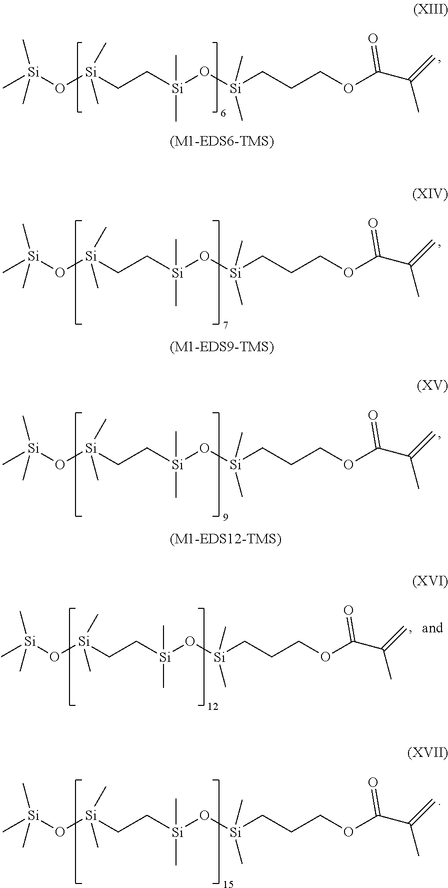

In an illustrative embodiment, as may be combined with one or more of the preceding paragraphs, the one or more ophthalmic device-forming silicone comonomers can include, as a class of representative ophthalmic device-forming silicone comonomers, one or more monomers of Formulas XVIII-XX:

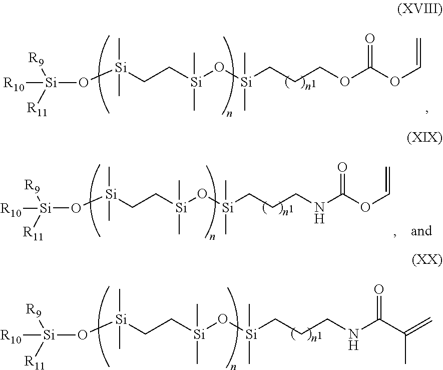

wherein R₉, R₁₀ and R₁₁ are independently hydrogen, an alkyl group, a haloalkyl group or other substituted alkyl groups and n and n¹ are as defined above.

In an illustrative embodiment, as may be combined with one or more of the preceding paragraphs, the one or more ophthalmic device-forming silicone comonomers can include, as a class of representative ophthalmic device-forming silicone comonomers, one or more monomers of Formulas XXI-XXIII:

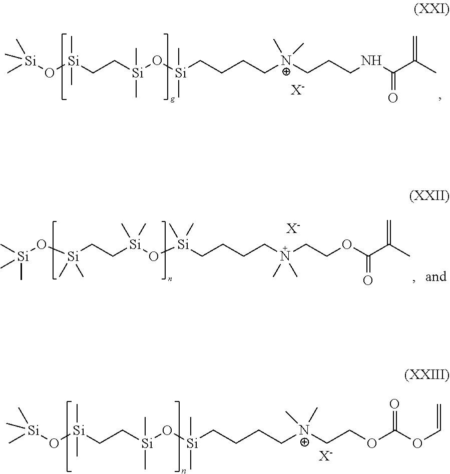

wherein n is as defined above and X⁻ is a counterion to provide an overall neutral charge.

Counterions capable of providing an overall neutral charge are well known to those of ordinary skill in the art and would include, for example, halide ions.

In an illustrative embodiment, as may be combined with one or more of the preceding paragraphs, the one or more ophthalmic device-forming silicone comonomers can include, as a class of representative ophthalmic device-forming silicone comonomers, one or more monomers of Formula XXIV:

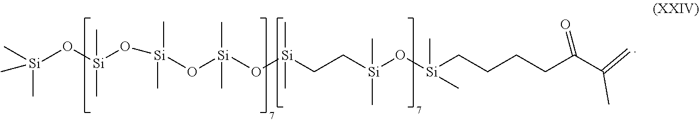

In an illustrative embodiment, as may be combined with one or more of the preceding paragraphs, the one or more ophthalmic device-forming silicone comonomers can include, as a class of representative ophthalmic device-forming silicone comonomers, one or more polysiloxane prepolymer represented by a structure of Formula XXV:

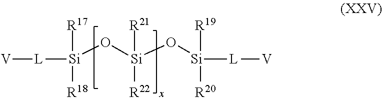

wherein each V is an independently reactive functional end group and includes, by way of example, a hydroxyl-containing reactive functional end group, and an amine-containing reactive functional end group, R¹⁷ to R²² are independently straight or branched, substituted or unsubstituted C₁-C₃₀ alkyl group, a substituted or unsubstituted C₃-C₃₀ cycloalkyl group, a substituted or unsubstituted C₄-C₃₀ cycloalkylalkyl group, a substituted or unsubstituted C₃-C₃₀ cycloalkenyl group, a substituted or unsubstituted C₆-C₃₀ aryl group, and a substituted or unsubstituted C₇-C₃₀ arylalkyl group, and L is independently a linking group.

A hydroxyl-containing reactive functional end group for use herein is a group of the general formula-OH. Representative examples of amine-containing reactive functional end groups for use herein include, by way of example, a (meth) acrylamide-containing reactive functional end group.

Linking group L is independently a straight or branched alkyl group, cycloalkyl group, an aryl group, an ether or polyether group, and an ester group as defined herein.

A representative example of a polysiloxane prepolymer is as follows:

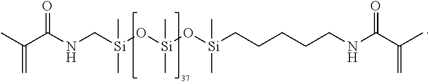

Methods for making the polysiloxane prepolymers described herein are well known and within the purview of one skilled in the art. In addition, the polysiloxane prepolymers are also commercially available from such sources as, for example, Gelest, Silar, Shin-Etsu, Momentive and Siltech.

Another class of representative ophthalmic device-forming silicone comonomers includes, for example, fluorinated monomers. Such monomers have been used in the formation of fluorosilicone hydrogels to reduce the accumulation of deposits on contact lenses made therefrom, as disclosed in, for example, U.S. Pat. Nos. 4,954,587; 5,010,141 and 5,079,319. Also, the use of silicone-containing monomers having certain fluorinated side groups, i.e., —(CF₂)—H, have been found to improve compatibility between the hydrophilic and silicone-containing monomeric units. See, e.g., U.S. Pat. Nos. 5,321,108 and 5,387,662.

The above silicone materials are merely exemplary, and other materials for use as substrates that have been disclosed in various publications and are being continuously developed for use in contact lenses and other biomedical devices can also be used. For example, a contact lens can be formed from at least a cationic monomer such as a cationic silicone-containing monomer or cationic fluorinated silicone-containing monomers.

Ophthalmic devices such as contact lenses can be manufactured employing various conventional techniques, to yield a shaped article having the desired posterior and anterior lens surfaces. Spincasting methods are disclosed in U.S. Pat. Nos. 3,408,429 and 3,660,545; and static casting methods are disclosed in U.S. Pat. Nos. 4,113,224, 4,197,266 and 5,271,876. Curing of the monomeric mixture may be followed by a machining operation in order to provide a contact lens having a desired final configuration. As an example, U.S. Pat. No. 4,555,732 discloses a process in which an excess of a monomeric mixture is cured by spincasting in a mold to form a shaped article having an anterior lens surface and a relatively large thickness. The posterior surface of the cured spincast article is subsequently lathe cut to provide a contact lens having the desired thickness and posterior lens surface. Further machining operations may follow the lathe cutting of the lens surface, for example, edge-finishing operations.

As one skilled in the art will readily appreciate, ophthalmic device surface functional groups of the ophthalmic device may be inherently present at the surface of the ophthalmic device. However, if the ophthalmic device contains too few or no functional groups, the surface of the ophthalmic device can be modified by known techniques or conventional functionalization with groups such as —OH, —NH₂ or —CO₂H. Suitable ophthalmic device surface functional groups of the biomedical device include a wide variety of groups well known to the skilled artisan. Representative examples of such functional groups include, but are not limited to, hydroxy groups, amino groups, carboxy groups, carbonyl groups, aldehyde groups, sulfonic acid groups, sulfonyl chloride groups, isocyanato groups, carboxy anhydride groups, lactone groups, azlactone groups, epoxy groups and groups being replaceable by amino or hydroxy groups, such as halo groups, or mixtures thereof. In an embodiment, the ophthalmic device surface functional groups of the ophthalmic device are amino groups and/or hydroxy groups.

Next, the resulting ophthalmic device such as a contact lens will be immersed in an aqueous packaging solution and stored in a packaging system according to illustrative embodiments disclosed herein. Generally, a packaging system for the storage of an ophthalmic device includes at least a sealed container containing one or more unused ophthalmic devices immersed in an aqueous packaging solution. In an illustrative embodiment, the sealed container is a hermetically sealed blister-pack, in which a concave well containing an ophthalmic device such as a contact lens is covered by a metal or plastic sheet adapted for peeling in order to open the blister-pack. The sealed container may be any suitable generally inert packaging material providing a reasonable degree of protection to the lens, preferably a plastic material such as polyalkylene, PVC, polyamide, and the like.

In accordance with one or more additional non-limiting illustrative embodiments, as may be combined with one or more of the preceding paragraphs, the aqueous packaging solution for use in the packaging system disclosed herein will contain at least hyaluronic acid or a salt or a derivative thereof and tremella. In illustrative embodiments, the aqueous packaging solution will contain from about 0.005% to about 5%, based on the total weight of the aqueous packaging solution, of hyaluronic acid or a salt or a derivative thereof. In illustrative embodiments, the aqueous packaging solution will contain from about 0.01% to about 1%, based on the total weight of the aqueous packaging solution, of hyaluronic acid or a salt or a derivative thereof. In illustrative embodiments, the aqueous packaging solution will contain from about 0.005% to about 5%, based on the total weight of the aqueous packaging solution, of tremella. In illustrative embodiments, the aqueous packaging solution will contain from about 0.01% to about 1%, based on the total weight of the aqueous packaging solution, of tremella.

Hyaluronic acid is a well-known, naturally occurring, water soluble biodegradable polymer composed of two alternatively linked sugars, D-glucuronic acid and N-acetylglucosamine, linked via alternating β-(1,4) and β-(1,3) glycosidic bonds. As mentioned hereinabove, hyaluronic acid is a glycosaminoglycan (GAG). In particular, hyaluronic acid is a non-sulfated GAG. The polymer is hydrophilic and highly viscous in aqueous solution at relatively low solute concentrations. It often occurs naturally as the sodium salt, sodium hyaluronate. Another salt of hyaluronic acid is potassium hyaluronate. Methods of preparing commercially available hyaluronan and salts thereof are well known. Hyaluronan can be purchased from Seikagaku Company, Clear Solutions Biotech, Inc., Pharmacia Inc., Sigma Inc., HTL Biotechnology, Contipro and Bloomage Biotechnology Corporation, and many other suppliers. Hyaluronic acid has repeating units of the structure represented by the following formula:

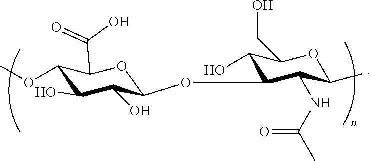

Accordingly, the repeating units in hyaluronic acid can be as follows:

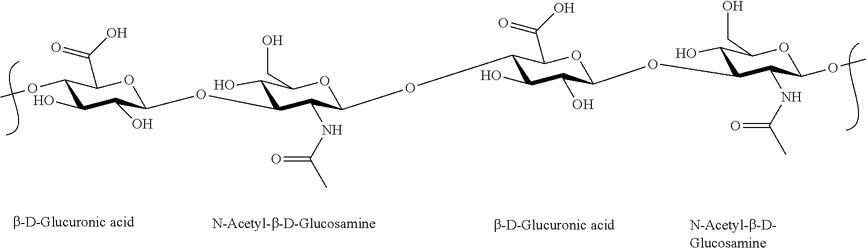

In general, hyaluronic acid or a salt thereof can have from about 2 to about 1,500,000 disaccharide units. In an embodiment, hyaluronic acid or a salt thereof can have a weight average molecular weight ranging from about 10,000 to about 4,000,000 Da in which the lower limit is from about 10,000, about 20,000, about 30,000, about 40,000, about 50,000, about 60,000, about 70,000, about 80,000, about 90,000, about 100,000 Da, about 200,000 Da, about 300,000 Da, about 400,000 Da, about 500,000 Da, about 600,000 Da, about 700,000 Da, about 800,000 Da, about 900,000 Da, about 1,000,000 Da, about 1,100,000 Da, about 1,200,000 Da, about 1,300,000 Da, about 1,400,000 Da, about 1,500,000 Da, and the upper limit is about 200,000, about 300,000, about 400,000, about 500,000, about 600,000, about 700,000, about 800,000, about 900,000, about 1,000,000, about 1,500,000 Da, about up to 2,000,000 Da, about up to 2,500,000 Da, about up to 2,800,000 Da, up to about 3,000,000 Da, up to about 3,200,000 Da, or up to about 4,0000,000 Da, where any of the lower limits can be combined with any of the upper limits.

The weight average molecular weight as disclosed herein is determined by size exclusion chromatography.

In accordance with one or more additional non-limiting illustrative embodiments, as may be combined with one or more of the preceding paragraphs, the hyaluronic acid or a salt thereof can be a derivative thereof, i.e., a modified hyaluronic acid or a salt thereof. For example, in an illustrative embodiment, the hyaluronic acid or a salt thereof can be a crosslinked polymeric network comprising a reaction product of hyaluronic acid or a salt thereof with another GAG, and one or more crosslinking agents, wherein the hyaluronic acid is different than the other glycosaminoglycan. Suitable GAGs that can be used to crosslink with hyaluronic acid include, for example, chondroitin sulfate (e.g., chondroitin 4- and 6-sulfates), heparan, heparin sulfate, heparosan, dermatan, dermatan sulfate, keratan sulfate, and other disaccharides such as sucrose, lactulose, lactose, maltose, trehalose, cellobiose, mannobiose and chitobiose. In an illustrative embodiment, the other GAG is chondroitin sulfate. Glycosaminoglycans can be purchased from Sigma, and many other biochemical suppliers such as HTL Biotechnology (France).

In an illustrative embodiment, the other GAGs for use herein can have a weight average molecular weight ranging from about 10,000 to about 3,000,000 Daltons (Da) in which the lower limit is from about 10,000, about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, or about up to 3,000,000 Da, where any of the lower limits can be combined with any of the upper limits.

Chondroitin sulfate is a linear sulfated polysaccharide composed of repeating β-D-glucuronic acid (GlcA) and N-acetyl-B-D-galactosamine (GalNAc) units arranged in the sequence by GlcA-β(1,3)-GalNAc-β(1,4) glycosidic bonds. In an embodiment, chondroitin sulfate has one or more repeating units of the structure represented by the following formula:

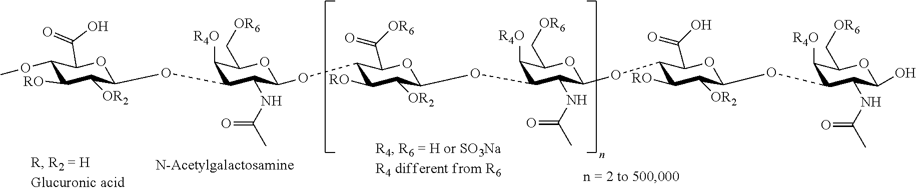

In an illustrative embodiment, chondroitin sulfate has repeating units of the structure represented by the following formula:

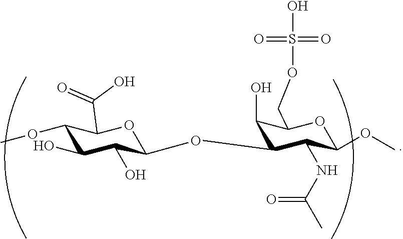

In general, chondroitin sulfate can have from about 2 to about 1,500,000 repeating units. In an embodiment, chondroitin sulfate can have a weight average molecular weight ranging from about 10,000 to about 3,000,000 Da in which the lower limit is from about 5,000, 10,000, about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, or about 3,000,000 Da where any of the lower limits can be combined with any of the upper limits or any of the upper limits can be combined with any of the upper limits.

In an illustrative embodiment, dermatan sulfate has repeating units of the structure represented by the following formula:

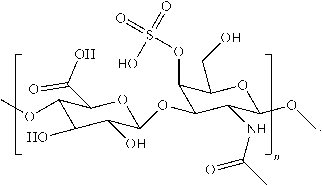

In general, dermatan sulfate can have from about 2 to about 1,500,000 repeating units. In an embodiment, dermatan sulfate can have a weight average molecular weight ranging from about 1,000 to about 2,000,000 Da in which the lower limit is from about 1,000, 5,000, 10,000, about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, or about 2,000,000 Da where any of the lower limits can be combined with any of the upper limits or any of the upper limits can be combined with any of the upper limits.

In an illustrative embodiment, heparin and heparin sulfate have repeating units of the structure represented by the following formula:

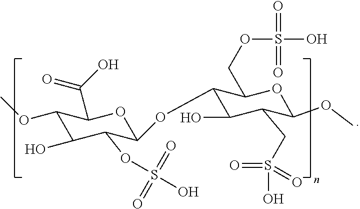

In general, heparin and heparin sulfate can have from about 2 to about 1,500,000 repeating units. In an embodiment, heparin and heparin sulfate can have a weight average molecular weight ranging from about 1,000 to about 3,000,000 Da in which the lower limit is from about 1,000, 5,000, 10,000, about 20,000, about 30,000, about 40,000, about 50,000, about 60,000, about 70,000, about 80,000, about 90,000, or about 100,000 Da, and the upper limit is about 40,000, 100,000, 200,000, about 300,000, about 400,000, about 500,000, about 600,000, about 700,000, about 800,000, about 900,000, about 1,000,000, or about 3,000,000 Da where any of the lower limits can be combined with any of the upper limits or any of the upper limits can be combined with any of the upper limits.

In an illustrative embodiment, keratan sulfate has repeating units of the structure represented by the following formula:

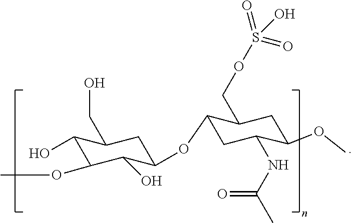

In general, keratan sulfate can have from about 2 to about 1,500,000 repeating units. In an embodiment, keratan sulfate can have a weight average molecular weight ranging from about 10,000 to about 3,000,000 Da in which the lower limit is from about 5,000, 10,000, about 20,000, about 30,000, about 40,000, about 50,000, about 60,000, about 70,000, about 80,000, about 90,000, or about 100,000 Da, and the upper limit is about 100,000, 200,000, about 300,000, about 400,000, about 500,000, about 550,000, 600,000, about 700,000, about 800,000, about 900,000, about 1,000,000, or about 3,000,000 Da where any of the lower limits can be combined with any of the upper limits or any of the upper limits can be combined with any of the upper limits.

The reaction product includes one or more crosslinking agents, e.g., to crosslink the hyaluronic acid or a salt thereof with the other glycosaminoglycan. The crosslinking agents for use herein can be any suitable crosslinking agent known in the art. In general, a suitable crosslinking agent is, for example, a crosslinking agent having complimentary functional groups to hyaluronic acid or a salt thereof and to the other glycosaminoglycan such as chondroitin sulfate. In an embodiment, a suitable crosslinking agent includes, for example, a bi- or polyfunctional crosslinking agent. The bi- or polyfunctional crosslinking agent connects the first glycosaminoglycan with the second glycosaminoglycan. In addition, the bi- or polyfunctional crosslinking agent further acts as a spacer between the first glycosaminoglycan and the second glycosaminoglycan. In general, the bi- or polyfunctional crosslinking agent comprises two or more functional groups capable of reacting with functional groups of the hyaluronic acid or a salt thereof and the other glycosaminoglycan such as chondroitin sulfate, resulting in the formation of covalent bonds.

Suitable bi- or polyfunctional crosslinking agents include, for example, divinyl sulfone, diepoxides, multiepoxides, dihydrazides, dihydric alcohols, polyhydric alcohols, polyhydric thiols, anhydrides, carbodiimdes, polycarboxylic acids, carboxymethyl thiols, cysteine, and cysteine-like amino acids and the like. In an embodiment, a bi- or polyfunctional crosslinking agent is a bis- or polyepoxide, such as diglycidyl ether derivatives. According to an embodiment, the bi- or polyfunctional epoxide crosslinking agent comprises two or more glycidyl ether functional groups. The glycidyl ether functional groups react with primary hydroxyl groups of the hyaluronic acid and the chondroitin sulfate, resulting in the formation of ether bonds. In an embodiment, suitable bis- or polyfunctional crosslinking agents include, for example, 1,4-butanediol diglycidyl ether (BDDE), 1,2-bis (2,3-epoxypropoxy) ethylene (EGDGE), ethylene glycol diglycidyl ether (EGDE), 1,2-cthanediol diglycidyl ether (EDDE), diepoxyoctane, 1,6-hexanediol diglycidyl ether, polypropylene glycol diglycidyl ether, polytetramethylene glycol diglycidyl ether, neopentyl glycol diglycidyl ether, polyglycerol polyglycidyl ester, diglycerol polyglycidyl ether, glycerol polyglycidyl ether, trimethylolpropane polyglycidyl ether, pentaerythritol polyglyglycidyl ether, sorbitol polyglycidyl ether, 1,2,7,8-diepoxyoctane, 1,3-butadiene diepoxide, pentaerythritol tetraglycidyl ether, polyepoxides and the like.

Suitable dihydrazide crosslinking agents include, for example, succinic acid dihydrazide, glutaric acid dihydrazide, adipic acid dihydrazide, pimelic acid dihydrazide, suberic acid dihydrazide, azalaic acid dihydrazide, sebacic acid dihydrazide, undecanedioic acid dihydrazide, dodecanedioic acid dihydrazide, brassylic acid dihydrazide, tetradecanedioic acid dihydrazide, pentadecanedioic acid dihydrazide, thapsic acid dihydrazide, octadecanedioic acid dihydrazide and the like.

Suitable dihydric alcohol crosslinking agents include, for example, ethylene glycol, propylene glycol, butylene glycol diethylene glycol, dipropylene glycol, neopentyl glycol, 1,3-propanediol, hexylene glycol, pentylene glycol, heptylene glycol, octylene glycol and the like. Suitable polyhydric alcohol crosslinking agents include, for example glycerin, pentaerythrite, xylitol, galactitol and the like. Suitable carbodiimide crosslinking agents include, for example, a compound of formula X—N═C═N—X, wherein each X independently is a C₁ to C₆ alkyl optionally substituted with 1-2 dialkylamino groups, or is a C₅ to C₆ cycloalkyl group, such as 1-ethyl-3-(3-dimethylaminopropyl) carbodimide hydrochloride, and cyclohexyl carbodiimide. Suitable anhydride crosslinking agents include, for example, methacrylic anhydride, octeyl succinic anhydride and the like. In an embodiment, a suitable crosslinking agent is an aldehyde crosslinking agent such as, for example, formaldehyde, gluteraldehyde, gluraraldehyde and the like. In an embodiment, a suitable crosslinking agent includes, for example, an acid chloride, n-hydroxysuccinimide, polyethylene glycol diacrylates, polyethylene glycol diamines, ureas, diisocyanates and the like.

The crosslinked polymeric network disclosed herein can be obtained by forming a solution of hyaluronic acid or a salt thereof and the other glycosaminoglycan, and adding the foregoing one or more crosslinking agents. The solution is stirred for a suitable time sufficient to crosslink at least hyaluronic acid or a salt thereof with the other glycosaminoglycan. In an embodiment, the crosslinking reaction can take place between 1° C. and about 99° C. over a time period of about 2 hours to about 24 hours.

The solution can contain a suitable solvent such as, for example, water, crown-ethers, dimethyl sulphoxide (DMSO), dimethyl formamide (DMF) and other aprotic solvents. In general, the amount of hyaluronic acid or a salt thereof can range from about 0.010 to about 50 wt. %, based on the total weight of the solution. In an embodiment, the amount of hyaluronic acid or a salt thereof can range from about 0.1 to about 5 wt. %, based on the total weight of the solution. In an embodiment, the amount of the other glycosaminoglycan can range from about 0.01 to about 50 wt. %, based on the total weight of the solution. In an embodiment, the amount of the other glycosaminoglycan can range from about 0.1 to about 5 wt. %, based on the total weight of the solution. The crosslinking agent can be added to the solution in an amount ranging from about 0.05 to about 10 wt. %, based on the total weight of the solution.

It will be readily understood and appreciated by those skilled in the art that the reaction product constitutes a complex mixture of compounds including, for example, hyaluronic acid or a salt thereof crosslinked with the other glycosaminoglycan, the hyaluronic acid or a salt thereof crosslinked with itself, the other glycosaminoglycan crosslinked itself, unreacted hyaluronic acid or a salt thereof and unreacted other glycosaminoglycan. For example, in an illustrative embodiment, hyaluronic acid or a salt thereof crosslinked with the other glycosaminoglycan can have a weight average molecular weight ranging from about 20,000 to about 6,000,000 Da in which the lower limit is from about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, about 2,000,000, about 3,000,000, about 4,000,000, about 5,000,000 or up to about 6,000,000 Da where any of the lower limits can be combined with any of the upper limits. It is not necessary to isolate one or more specific components of the reaction product mixture. Indeed, the reaction product mixture can be employed as is.

In an illustrative embodiment, as may be combined with one or more of the preceding paragraphs, the crosslinked polymeric network disclosed herein can be further crosslinked with one or more additional glycosaminoglycans. In general, the one or more additional glycosaminoglycans can be any of the glycosaminoglycans discussed hereinabove. In an embodiment, the one or more additional glycosaminoglycans are hyaluronic acid. In an embodiment, the one or more additional glycosaminoglycans are chondroitin sulfate. In an embodiment, the one or more additional glycosaminoglycans include hyaluronic acid and chondroitin sulfate.

The crosslinking agent for crosslinking the one or more of the same or different additional glycosaminoglycans with the foregoing crosslinked polymeric network can be any of the crosslinking agents discussed above. As one skilled in the art will understand, the crosslinking agent can be the same crosslinking agent or a different crosslinking agent as the crosslinking agent used in the foregoing reaction product.

In general, the crosslinked polymeric network disclosed can be further crosslinked with one or more of the same or different additional glycosaminoglycans in substantially the same manner as discussed above.

In another illustrative embodiment, a derivatized hyaluronic acid or a salt thereof can be obtained by further reacting the hyaluronic acid or a salt thereof with one or more phospholipids and optionally one or more crosslinking agents to form glycophospholipid polymers or crosslinked glycophospholipid polymeric networks. Suitable phospholipids for reacting with the one or more GAGs comprise, for example, phosphorylcholine, phosphorylhistidine, phosphorylproline, phosphorylserine, α-phosphatidylcholine, α-phosphatidylethanolamine, α-phosphatidyl-L-serine, α-phosphatidylinositol, α-phosphatidic acid, α-phosphatidyl-DL-glycerol, α-lysophosphatidylcholine, sn-glycero-3-phosphatidylcholine (GPC) (also choline alfoscerate) derived from soybean phosphatidylcholine sphingomyelin, and cardiolipin. The phospholipids described herein are either commercially available or can be made by techniques well known in the art, see, e.g., Park et al., “Facile Syntheses of L-α-Glycerophosphorylcholine”, Bull. Korean Chem. Soc. 2010, Vol. 31, No. pp. 9 2689-2691.

In an embodiment, a phosphorylcholine-containing phospholipid comprises a zwitterionic group as follow:

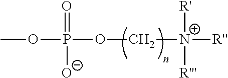

wherein n is an integer of 1 to 5 and R′, R″ and R′″ independently of each other are a C₁ to C₈ alkyl group, a C₁ to C₈ hydroxyalkyl group or a hetero atom-containing group such as histidine and proline. As one skilled in the art will readily appreciate, the foregoing phosphorylcholine-containing phospholipid will further include one or more reactive groups capable of reacting with the hyaluronic acid or a salt thereof, e.g., hydroxyl-containing groups, epoxide-containing groups, amine-containing groups, aldehyde-containing groups, thiol-containing groups, carboxylic acid-containing groups, cyano-containing groups, halogen-containing groups, etc. Representative examples of suitable phosphorylcholine phospholipids having the foregoing zwitterionic group include, but are not limited to, glycerylphosphorylcholine, ethanaminium, 2·[hydroxy·2·oxiranylmethoxy] phosphinyl)oxy)·N,N,N·trimethyl, inner salt, and hydroxyethylphophorylcholine.

The term “hydroxyl-containing group” should be understood to mean any group which contains a hydroxyl moiety. Representative examples of hydroxyl-containing groups for use herein include, by way of example, a hydroxy group attached directly to the rest of the molecule, i.e., —OH, or one or more hydroxy-containing groups attached to the rest of the molecule via a linking group, e.g., an alkyl, cycloalkyl, cycloalkylalkyl, cycloalkenyl, aryl or an arylalkyl group and the like.

The term “epoxide-containing group” should be understood to mean any group which contains an epoxide moiety. Representative examples of epoxide-containing groups for use herein include, by way of example, alkylene oxides and in particular lower alkylene oxides such as ethylene oxide, propylene oxide, butylene oxide, alcohol epoxides such as glycidol, and epihalohydrins such as epichlorohydrin, epibromohydrin, epiiodohydrin, 1,2-epoxy-4-chlorobutane, 1,2-epoxy-4-bromobutane, 1,2-epoxy-4-iodobutane, 2,3-epoxy-4-chlorobutane, 2,3-epoxy-4-bromobutane, 2,3-epoxy-4-iodobutane, 2,3-epoxy-5-chloropentane, 2,3-epoxy-5-bromopentane, 1,2-epoxy-5-chloropentane, etc.; epoxy compounds such as 2,2-bis (p-1,2-epoxypropoxyphenyl)-propane, 1,4-bis (1,2-epoxypropoxy) benzene, N,N′-bis(2,3-epoxypropyl) piperazine, etc.

The term “amine-containing group” should be understood to mean any group which contains an amine moiety. Representative examples of amine-containing groups for use herein include, by way of example, an alky or arylamine group such as an amine of the general formula —R¹NR²R³ wherein R¹ is a C₂ to C₃₀ alkylene, arylene, or cycloalkylene and R² and R³ are independently C₁ to C₃₀ hydrocarbons such as, for example, alkyl groups, aryl groups, or cycloalkyl groups as defined herein.

The term “aldehyde-containing group” should be understood to mean any group which contains an aldehyde moiety. Representative examples of aldehyde-containing groups for use herein include, by way of example, an aldehyde group attached directly to the rest of the molecule, i.e., —CHO, or one or more aldehyde groups attached to the rest of the molecule via a linking group, e.g., an alkylene, cycloalkyl, cycloalkylalkylene, cycloalkenyl, aryl or an arylalkylene group and the like.

The term “thiol-containing group” should be understood to mean any group which contains a thiol moiety. Representative examples of thiol-containing groups for use herein include, by way of example, a thiol group attached directly to the rest of the molecule, i.e., —SH, or one or more thiol groups attached to the rest of the molecule via a linking group, e.g., an alkylene, cycloalkyl, cycloalkylalkylene, cycloalkenyl, aryl or an arylalkylene group and the like.

The term “carboxylic acid-containing group” should be understood to mean any group which contains a carboxylic acid moiety. Representative examples of carboxylic acid-containing groups for use herein include, by way of example, a carboxylic acid group attached directly to the rest of the molecule, i.e., —COOH, or one or more carboxylic acid groups attached to the rest of the molecule via a linking group, e.g., an alkylene, cycloalkyl, cycloalkylalkylene, cycloalkenyl, aryl or an arylalkylene group and the like.

The term “cyano-containing group” should be understood to mean any group which contains a cyano moiety. Representative examples of cyano-containing groups for use herein include, by way of example, a cyano group attached directly to the rest of the molecule, i.e., —CN, or one or more cyano groups attached to the rest of the molecule via a linking group, e.g., an alkylene, cycloalkyl, cycloalkylalkylene, cycloalkenyl, aryl or an arylalkylene group and the like.

The term “halogen-containing group” should be understood to mean any group which contains a halogen moiety. Representative examples of halogen-containing groups for use herein include, by way of example, a halogen group attached directly to the rest of the molecule, i.e., F, Cl, B, I and the like, or one or more halogen groups attached to the rest of the molecule via a linking group, e.g., an alkylene, cycloalkyl, cycloalkylalkylene, cycloalkenyl, aryl or an arylalkylene group and the like.

Representative examples of alkyl groups for use herein include, by way of example, a straight or branched hydrocarbon chain radical containing carbon and hydrogen atoms of from 1 to about 18 carbon atoms with or without unsaturation, to the rest of the molecule, e.g., methyl, ethyl, n-propyl, 1-methylethyl (isopropyl), n-butyl, n-pentyl, etc., and the like.

Representative examples of alkylene groups for use herein include, by way of example, a straight or branched alkyl chain radical containing carbon and hydrogen atoms of from 1 to about 30 carbon atoms or from 1 to about 6 carbon atoms with or without unsaturation, to the rest of the molecule, e.g., methylene, ethylene, and the like.

Representative examples of cycloalkyl groups for use herein include, by way of example, a substituted or unsubstituted non-aromatic mono or multicyclic ring system of about 3 to about 18 carbon atoms such as, for example, cyclopropyl, cyclobutyl, cyclopentyl, cyclohexyl, perhydronapththyl, adamantyl and norbornyl groups bridged cyclic group or sprirobicyclic groups, e.g., sprio-(4,4)-non-2-yl and the like, optionally containing one or more heteroatoms, e.g., O and N, and the like.

Representative examples of cycloalkylalkyl groups for use herein include, by way of example, a substituted or unsubstituted cyclic ring-containing radical containing from about 3 to about 18 carbon atoms directly attached to the alkyl group which are then attached to the main structure of the monomer at any carbon from the alkyl group that results in the creation of a stable structure such as, for example, cyclopropylmethyl, cyclobutylethyl, cyclopentylethyl and the like, wherein the cyclic ring can optionally contain one or more heteroatoms, e.g., O and N, and the like.

Representative examples of cycloalkenyl groups for use herein include, by way of example, a substituted or unsubstituted cyclic ring-containing radical containing from about 3 to about 18 carbon atoms with at least one carbon-carbon double bond such as, for example, cyclopropenyl, cyclobutenyl, cyclopentenyl and the like, wherein the cyclic ring can optionally contain one or more heteroatoms, e.g., O and N, and the like.

Representative examples of aryl groups for use herein include, by way of example, a substituted or unsubstituted monoaromatic or polyaromatic radical containing from about 5 to about 25 carbon atoms such as, for example, phenyl, naphthyl, tetrahydronapthyl, indenyl, biphenyl and the like, optionally containing one or more heteroatoms, e.g., O and N, and the like.

Representative examples of arylalkylene groups for use herein include, by way of example, a substituted or unsubstituted aryl group as defined above directly bonded to an alkylene group as defined above, e.g., —CH₂C₆H₅, —C₂H₅C₆H₅ and the like, wherein the aryl group can optionally contain one or more heteroatoms, e.g., O and N, and the like.

The phospholipids for use herein are either commercially available or can be prepared by methods known in the art. For example, ethanaminium, 2·[[hydroxy-2-oxiranylmethoxy]phosphinyl)oxy)-N,N,N·trimethyl, inner salt can be prepared according to a method described in Example 11 in Lindberg, et al., “Efficient Synthesis of Phospholipids from Glycidyl Phosphates”, J. Org. Chemistry, 2002, 67, pp. 194-199, or as in U.S. Pat. No. 7,674,782, the contents of which are incorporated by reference herein. In addition, hydroxyethylphosphorylcholine can be prepared according to Zhang et al., “Microscale NMR Screening of New Detergents for Membrane Protein Structural Biology”, Journal of the American Chemical Society, 2008, 130 (23), pp. 7357-7363, the contents of which are incorporated by reference herein, and is also commercially available from Shanghai Chemhere Co., Ltd. (Shanghai, Hong Kong).

In an illustrative embodiment, as may be combined with one or more of the preceding paragraphs, the reaction product of the hyaluronic acid or a salt thereof and the one or more phospholipids includes one or more crosslinking agents, e.g., to crosslink the hyaluronic acid or a salt thereof with the one or more phospholipids and form a crosslinked glycophospholipid polymeric network. The crosslinking agents for use herein can be any of the crosslinking agent discussed above.

The glycophospholipid polymeric network disclosed herein can be obtained by forming a solution of the hyaluronic acid or a salt thereof and the one or more phospholipids, and optionally adding the one or more crosslinking agents when forming a crosslinked polymeric network. The solution is stirred for a suitable time sufficient to react and/or crosslink the hyaluronic acid or a salt thereof and the one or more phospholipids. In an embodiment, the reaction between the hyaluronic acid or a salt thereof and the one or more phospholipids can take place between 1° C. and about 99° C., over a time period of about 2 hours to about 36 hours.

The solution can contain suitable one or more solvents such as, for example, water, crown-ethers, dimethyl sulphoxide (DMSO), dimethyl formamide (DMF) and other aprotic solvents.

In general, the amount of the hyaluronic acid or a salt thereof in the solution can range from about 0.010 to about 50 wt. %, based on the total weight of the solution. In an illustrative embodiment, the amount of the hyaluronic acid or a salt thereof in the solution can range from about 0.1 to about 5 wt. %, based on the total weight of the solution. In an illustrative embodiment, the amount of the one or more phospholipids in the solution can range from about 0.01 to about 50 wt. %, based on the total weight of the solution. In an illustrative embodiment, the amount of the one or more phospholipids in the solution can range from about 0.1 to about 5 wt. %, based on the total weight of the solution. The crosslinking agent can be added to the solution in an amount ranging from about 0.05 to about 20 wt. %, based on the total weight of the solution.

It will be readily understood and appreciated by those skilled in the art that the reaction product constitutes a complex mixture of compounds. For example, a reaction product of hyaluronic acid or a salt thereof, one or more phospholipids, and one or more crosslinking agents can include, by way of example, the polymer obtained from the reaction between hyaluronic acid or a salt thereof and the one or more phospholipids, the hyaluronic acid or a salt thereof crosslinked with the phospholipid, the hyaluronic acid or a salt thereof crosslinked with itself, the phospholipid crosslinked with itself, unreacted hyaluronic acid or a salt thereof and unreacted phospholipid. In an illustrative embodiment, the hyaluronic acid or a salt thereof crosslinked with a phospholipid can have a weight average molecular weight ranging from about 20,000 to about 6,000,000 Da in which the lower limit is from about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, about 2,000,000, about 3,000,000, about 4,000,000, about 5,000,000 or up to about 6,000,000 Da where any of the lower limits can be combined with any of the upper limits. It is not necessary to isolate one or more specific components of the reaction product mixture. Indeed, the reaction product mixture can be employed as is.

In an illustrative embodiment, as may be combined with one or more of the preceding paragraphs, a derivatized hyaluronic acid or a salt thereof can include hyaluronic acid or a salt thereof having a polymer backbone and one or more side chains comprising a polyalkylene glycol-containing residue grafted onto the polymer backbone to form a grafted hyaluronic acid polymer. In general, the hyaluronic acid or a salt thereof will have a reactive functional group in the polymer backbone for grafting a polymer comprising polyalkylene glycol chains and at least one reactive end group or a salt thereof. Suitable reactive functional groups in the polymer backbone include carboxylate-containing groups, hydroxyl-containing groups, and other groups including polymerizable functionalities such as allylic, vinylic, acrylate, methacylate, methacrylamide etc. The hyaluronic acid or a salt thereof for use herein can have a weight average molecular weight as discussed above.

The polyalkylene glycol-containing residue grafted onto a reactive functional group in the polymer backbone of the hyaluronic acid or a salt thereof is derived from a polymer comprising polyalkylene glycol chains and at least one reactive end group or a salt thereof (e.g., HCl). The polyalkylene glycol chains can range from 2 to 10,000 subunits or from 2 to 5000 subunits. In an embodiment, the polyalkylene glycol chains comprise a structure: —((CH₂)ₐ—O)_{b}— where “a” is from 2 to 6 or from 2 to 4 and “b” is from 2 to 10,000 or from 2 to 5000. In an illustrative embodiment, a polyalkylene glycol is one or more of polyethylene glycol chains such as (e.g., —(CH₂CH₂O)_{b}—) (i.e., PEG), polypropylene glycol chains (e.g., —(CH₂CH₂CH₂O)_{b}—), polybutylene glycol chains (e.g., —(CH₂CH₂CH₂CH₂O)_{b}—), ethylene oxide-propylene oxide chains, and ethylene oxide-butylene oxide chains.

The at least one reactive end group of the polymer comprising polyalkylene glycol chains includes a reactive functional group capable of grafting on to the reactive functional group in the polymer backbone of the hyaluronic acid or a salt thereof. Suitable reactive functional groups include, for example, a halogen, amino groups, aldehyde groups, carboxylic acid groups, alcohol groups, thiol groups, hydrazide groups, glycidyl groups, etc. These groups are attached to the polymeric compound by way of a linker group “X”. Examples of reactive functional groups include —X—PDMS—NH₂ where PDMS is polydimethylsiloxane having a number molecular weight ranging from about 100 to about 150,000 Da, —X—OH, —X—NH₂, —X—SH, and —X—C(O)—R′ where R′ is hydrogen or an organic hydrocarbyl moiety comprised of 1 to 20 carbon atoms such as a lower alkyl group (e.g., methyl, ethyl, propyl, etc.) or benzyl.

Suitable linker groups “X” for attaching the reactive functional end group to the polymer include, for example, any of the following: —C(O)—, —N—C(O)—NH—CH₂—, —N—C(O)—NH—CH₂—CH₂—, —CH₂—, —CH₂—CH₂—, —CH₂—CH₂—CH₂—, —CH₂—CH₂—CH₂—CH₂—, —CH₂—O—CH₂—, —CH₂—O—CH₂—CH₂—, —CH₂—CH₂—CH₂—O—CH₂—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—(Si—O—Si)ₙ—O—Si—CH₂—CH₂—CH₂— where n=3 to 100 and combinations of two or more of any of the foregoing.

The other end group can be either an inert end-capping group or a reactive end-group. An inert end-capping group is one that does not readily undergo chemical transformation under typical synthetic reaction conditions. A reactive end group can be used for further cross-linking. Suitable end-capping groups include, for example, an alkoxy group, a hydroxyl group, a thiol group, an amine group and an ethylenically polymerizable group such as, for example, an acrylate or methacrylate group. An alkoxy group is represented by the general formula-OR, where R is an organic moiety comprised of 1-20 carbon atoms such as a lower alkyl group (e.g., methyl or ethyl) or benzyl. R, however, may be saturated or unsaturated, and includes aryl, heteroaryl, cyclo, heterocyclo, and substituted forms of any of the foregoing. For example, an end-capped PEG can comprise the structure RO—(CH₂CH₂O)ₙ— where R is as defined above. In an illustrative embodiment, suitable end groups include, by way of example, —OCH₃, —OCH₂CH₃, —OCH₂(C₆H₅), —NH₂, —OH, and —SH.

The polymers comprising polyalkylene glycol chains and at least one reactive end group or a salt thereof for use herein include polymers having a variety of molecular weights, structures or geometries (e.g., branched, linear, and the like). In an illustrative embodiment, the weight average molecular weight of a polymer comprising polyalkylene glycol chains and at least one reactive end group or a salt thereof may range from about 100 Da to about 10,000 Da. For example, in an illustrative embodiment, the weight average molecular weight of a polymer comprising polyalkylene glycol chains and at least one reactive end group or a salt thereof can be greater than about 100 Daltons, or greater than about 250 Da, or greater than about 500 Da, or greater than about 750 Da, or greater than about 1,000 Da, or greater than about 2,000 Da, or greater than about 5,000 Da, or greater than about 7,500 Da. In another illustrative embodiment, the weight average molecular weight of a polymer comprising polyalkylene glycol chains and at least one reactive end group or a salt thereof can be less than about 10,000 Da, or less than about 7,500 Da, or less than about 5,000 Da, or less than about 2,000 Da, or less than about 1,000 Da, or less than about 750 Da, or less than about 600 Da. As one skilled in the art can appreciate, any molecular weight between those listed above can be used.

The foregoing polymers are either commercially available from various sources such as BroadPharm, Sigma, JenKem, and Advanced Polymer Materials Inc. or can be prepared according to methods well known in the art.

In an illustrative embodiment, a polymer comprising polyalkylene glycol chains and at least one reactive end group or a salt thereof is a polymer or a salt thereof having the following structure:

Z—(((CH₂)ₐ—O)_{b})_{c}—Y

wherein Z is an end-capped group, Y is a reactive functional group, a is from 2 to 6, b is from 2 to 10,000 and c is 1 or 2.

Z is an end-capped (or end-capping) group which can be an inert group or a reactive group present on a terminus of the polymeric compound such as a polyethylene glycol (PEG) polymer. Suitable end-capped groups include any of those discussed above. Y is a reactive functional group capable of grafting on to the reactive functional group in the polymer backbone of the GAG. Suitable reactive functional groups include any of those discussed above. Suitable linker groups “X” for attaching the reactive functional group any of those discussed above.

The polymers may be derived from a polyalkylene glycol. In general, a polyalkylene glycol comprises the following structure: —((CH₂)ₐ—O)_{b}— where “a” is from 2 to 6 or from 2 to 4 and “b” is from 2 to 10,000 or from 2 to 5000. In an illustrative embodiment, a polyalkylene glycol is one or more of a polyethylene glycol (e.g., —(CH₂CH₂O)_{b}—), a polypropylene glycol (e.g., —(CH₂CH₂CH₂O)_{b}—) a polybutylene glycol (e.g., —(CH₂CH₂CH₂CH₂O)_{b}—), ethylene oxide-propylene oxide, and ethylene oxide-butylene oxide. The polyalkylene glycols for use herein include polyalkylene glycols having a variety of molecular weights, structures or geometries (e.g., branched, linear, and the like) as discussed above.

In an embodiment, representative examples of such polymers for use herein include any of the following:

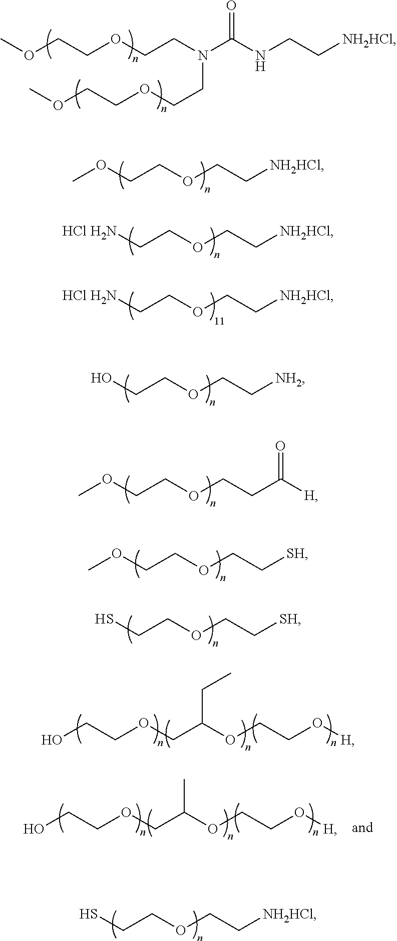

wherein n is from 2 to 10,000.

The grafted hyaluronic acid polymers disclosed herein can be obtained by grafting the reactive functionality of the one or more polymers comprising polyalkylene glycol chains onto the reactive functionality in the polymer backbone of the hyaluronic acid or a salt thereof. For example, in an illustrative embodiment, an amine reactive end group of the polymer comprising polyalkylene glycol chains can be grafted onto a carboxylic acid group in the polymer backbone of the glycosaminoglycan. The graft polymerization reaction can obtain a degree of grafting, i.e., the number of sidechains in the polymer backbone containing the polyalkylene glycol-containing residue, ranging from about 5 to about 100%. In an illustrative embodiment, the degree of grafting can range from about 10 to about 90%. In an illustrative embodiment, the degree of grafting can range from about 20 to about 80%.

In an illustrative embodiment, the hyaluronic acid or a salt thereof can be added to the reaction mixture in an amount ranging from about 0.05 wt. % to about 10 wt. %, based on the total weight of the reaction mixture. In an illustrative embodiment, the hyaluronic acid or a salt thereof can be added to the reaction mixture in an amount ranging from about 0.5 wt. % to about 5 wt. %, based on the total weight of the reaction mixture.

In an embodiment, a polymer comprising polyalkylene glycol chains can be added to the reaction mixture in an amount ranging from about 0.01 wt. % to about 20 wt. %, based on the total weight of the reaction mixture. In an illustrative embodiment, a polymer comprising polyalkylene glycol chains can be added to the reaction mixture in an amount ranging from about 0.10 wt. % to about 0.5 wt. %, based on the total weight of the reaction mixture.

The grafting reaction is ordinarily carried out in the presence of a catalyst system. In some embodiments, the catalyst system is a carbodiimide catalyst system such as, for example, 1-ethyl-3-(3-dimethylaminopropyl) carbodiimide (EDC). In some embodiments, a co-catalyst is used with the carbodiimide catalyst system. Suitable co-catalysts include, for example, hydroxybenzotriazole (HOBt), N-hydroxysuccinimide (NHS) and sulfo-N-hydroxysuccinimide (Sulfo-NHS). In some embodiments, the catalyst system includes EDC/NHS. In an embodiment, the EDC is added to the reaction mixture in an amount ranging from about 0.01 wt. % to about 20 wt. %, based on the total weight of the reaction mixture. In an embodiment, the NHS is added to the reaction mixture in an amount ranging from about 0.01 wt. % to about 20 wt. %, based on the total weight of the reaction mixture.

In an illustrative embodiment, the grafting reaction can be carried out by reacting the hyaluronic acid or a salt thereof with the polymer under suitable grafting conditions using a catalyst system such as EDC/NHS or EDC/HOBt coupling at a pH of about 6.8 with about 1 to about 5 weight percent dissolved solids in water to form random copolymers or block copolymers.

In another embodiment, the grafting reaction is carried out by reacting the hyaluronic acid or a salt thereof with monomers capable of forming a polymer comprising polyalkylene glycol chains and at least one reactive end group or a salt thereof in-situ. For example, the reaction can be carried out by first forming a solution containing at least the hyaluronic acid or a salt thereof and cocatalyst system. Next, the hyaluronic acid or a salt thereof is activated by adding an activator to the solution. A suitable activator includes, for example, one or more epoxyamines. Epoxyamines are molecules that generally include both at least one amine moiety (e.g., a primary, secondary, tertiary, or quaternary amine) and at least one epoxide moiety. The epoxyamine compound can be a monoepoxyamine compound and or a polyepoxyamine compound, i.e., an epoxyamine containing one or more amine groups and one or more epoxide groups. In an embodiment, a suitable epoxyamine compound is one in which the amine moiety is linked to the epoxide moiety by way of a C₁ to C₃₀ alkylene group. Suitable epoxyamine compounds include, for example, epoxyethylamine, epoxypropylamine, epoxybutylamine, epoxyamyl amine and the like. The activation reaction can be carried out at a suitable temperature and for a time period to react the activator with the glycosaminoglycan, e.g., at room temperature, and a time period ranging from about 10 hours to about 48 hours. In an embodiment, an epoxyamine can be added to the reaction mixture in an amount ranging from about 0.01 to about 50 wt. %, based on the total weight of the reaction mixture.

After the activator has been reacted with the hyaluronic acid or a salt thereof, the monomers capable of forming in-situ a polymer comprising polyalkylene glycol chains and at least one reactive end group or a salt thereof are added to the reaction mixture. In an embodiment, the monomers include a polyol and an epoxy alcohol. Suitable polyols include, for example one or more diols. Representative diols include, by way of example, a C₂ to C₁₂ diol such as ethylene glycol, propylene glycol, 1,3-propanediol, 1,2-butanediol, 1,4-butanediol, 2-methyl-1,3-propanediol, poloxamer 407 and the like. The epoxyalcohol compound can be a monoepoxyalcohol compound and or a polyepoxyalcohol compound, i.e., an epoxyalcohol containing one or more alcohol groups and one or more epoxide groups. In an embodiment, a suitable epoxyalcohol compound is one in which the alcohol moiety is linked to the epoxide moiety by way of a C₁ to C₃₀ alkylene and or alkyne group. Suitable epoxyalcohol compounds include, for example, glycidyl alcohol, 3-oxiranyl-2-Propen-1-ol, 3-(2-oxiranyl) 2-propen-1-ol, 1-(2,3-dihydroxypropyl) 4-(2-oxiranylmethyl) ester of 2-butenedioic acid, and a 1-(2-hydroxyethyl) 2-(2-oxiranylmethyl) ester of 1,2-benzenedicarboxylic acid.

In general, the polyol and epoxyalcohol can be added sequentially or simultaneously to the reaction mixture. In an embodiment, the polyol is added to the reaction mixture and reacted with the activated hyaluronic acid or a salt thereof, followed by the polyol to form the polyalkylene glycol-containing residue. The reaction can be carried out at a suitable temperature and for a time period for the completion of the reaction to maximize the yield of the product polyalkylene glycol residue onto the polymer backbone of the glycosaminoglycan, e.g., at room temperature and a time period ranging from about 10 hours to about 48 hours. In an embodiment, a polyol can be added to the reaction mixture in an amount ranging from about 0.01 to about 50 wt. %, and an epoxyalcohol can be added to the reaction mixture in an amount ranging from about 0.01 to about 50 wt. %, based on the total weight of the reaction mixture.

The resulting grafted hyaluronic acid polymer can be a random copolymer or a block copolymer. In an illustrative embodiment, a grafted hyaluronic acid polymer disclosed herein can have a weight average molecular weight ranging from about 20,000 to about 6,000,000 Da in which the lower limit is from about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, about 2,000,000, about 3,000,000, about 4,000,000, about 5,000,000 or up to about 6,000,000 Da where any of the lower limits can be combined with any of the upper limits.

In another embodiment, a crosslinked polymer network can be formed by either reacting the foregoing grafted hyaluronic acid polymers with one or more crosslinking agents, or adding one or more crosslinking agents to the grafting reaction mixture. The crosslinking agents for use herein can be any of the crosslinking agents discussed above such as, for example, a bi- or polyfunctional crosslinking agent comprising two or more functional groups capable of reacting with functional groups of the grafted hyaluronic acid polymers resulting in the formation of covalent bonds.

In an embodiment, a crosslinked polymeric network described in exemplary embodiments herein can be obtained by forming a solution of the one or more grafted hyaluronic acid polymers and adding one or more of the foregoing crosslinking agents. In an embodiment, a crosslinked polymeric network described in exemplary embodiments herein can be obtained by adding one or more of the foregoing crosslinking agents to the grafting solution of the one or more hyaluronic acid polymers and one or more polymers. The solution is stirred for a suitable time sufficient to crosslink the reaction mixture. In an embodiment, the crosslinking can take place between 1° C. and about 99° C. over a time period of about 2 hours to about 48 hours.

The solution can contain a suitable solvent such as, for example, water, crown-ethers, dimethyl sulphoxide (DMSO), dimethyl formamide (DMF) and other aprotic solvents. The pH of the solution can be adjusted if necessary by adding, for example, a hydroxide such as sodium hydroxide. In general, the crosslinking agent can be added to the solution in an amount ranging from about 0.01 wt. % to about 10 wt. %, based on the total weight of the solution. When crosslinking the grafted hyaluronic acid polymer, the amount of the grafted hyaluronic acid polymer can range from about 0.010 wt. % to about 50 wt. %, based on the total weight of the solution. In an embodiment, the amount of the grafted hyaluronic acid polymer can range from about 0.01 wt. % to about 5 wt. %, based on the total weight of the solution.

In an embodiment, one or more other glycosaminoglycans can be added to the reaction of the grafted hyaluronic acid polymers and one or more crosslinking agents to form a crosslinked polymeric network, i.e., to crosslink the grafted hyaluronic acid polymers with the one or more glycosaminoglycans. In general, the one or more glycosaminoglycans can be any of the glycosaminoglycans discussed hereinabove. In an embodiment, the one or more glycosaminoglycans are hyaluronic acid or a salt thereof. In an embodiment, the one or more glycosaminoglycans are chondroitin sulfate. In an embodiment, the one or more glycosaminoglycans include hyaluronic acid or a salt thereof and chondroitin sulfate. In an illustrative embodiment, the amount of the one or more glycosaminoglycans can range from about 0.010 wt. % to about 50 wt. %, based on the total weight of the solution. In an embodiment, the amount of the one or more glycosaminoglycans can range from about 0.01 wt. % to about 5 wt. %, based on the total weight of the solution.

The one or more crosslinking agents will have complimentary functional groups to the grafted hyaluronic acid polymer and to the glycosaminoglycan. For example, a suitable crosslinking agent such as a bi- or polyfunctional crosslinking agent connects the grafted hyaluronic acid polymer with the glycosaminoglycan, and further acts as a spacer between the grafted hyaluronic acid polymer and the glycosaminoglycan.

It will be readily understood and appreciated by those skilled in the art that the reaction product constitutes a complex mixture of compounds including, for example, the grafted hyaluronic acid polymer crosslinked with the glycosaminoglycan, the grafted hyaluronic acid polymer crosslinked with the grafted hyaluronic acid polymer, the glycosaminoglycan crosslinked with the glycosaminoglycan, unreacted grafted hyaluronic acid polymer and unreacted glycosaminoglycan. For example, in an illustrative embodiment, a grafted glycosaminoglycan polymer crosslinked with a glycosaminoglycan can have a weight average molecular weight ranging from about 20,000 to about 6,000,000 Da in which the lower limit is from about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, about 2,000,000, about 3,000,000, about 4,000,000, about 5,000,000 or up to about 6,000,000 Da where any of the lower limits can be combined with any of the upper limits. It is not necessary to isolate one or more specific components of the reaction product mixture. Indeed, the reaction product mixture can be employed as is. If necessary, any excess crosslinker can be removed by dialysis or precipitation in ethanol.

In accordance with one or more additional non-limiting illustrative embodiments, as may be combined with one or more of the preceding paragraphs, the aqueous packaging solution for use in the packaging systems disclosed herein further contains tremella. In an illustrative embodiment, tremella is a polysaccharide which comprises or is extracted from the mushroom Tremella species. For example, tremella is a polysaccharide which comprises or is extracted from the mushroom species selected from the group consisting of Tremella fuciformis, Tremella mesenterica, Tremella aurantia, mixtures thereof, and combinations thereof.

Tremella can also be obtained from a fermentation process. In an illustrative embodiment, tremella is a polysaccharide which is obtained from a fermentation process. For example, dextrose and mannose are fermented together in a bioreactor and vertical bowl-type centrifuge for cell separation followed by washing and filtration. Purification by column chromatography and drying after solvent evaporation yields tremella with a main chain of TFBP-A which is composed of (1→3)-D-Man-, and the side chain is composed of (1→3,6)-D-Man, (1→6)-D-GlcN, (1-+4,6)-D-GalA, (1→6)-D-Gal, (1→)-L-Rib, (1→)-L-Xyl and (1-+3)-L-Fuc, the degree of branching of polysaccharides is 61.55%.

In accordance with one or more additional non-limiting illustrative embodiments, as may be combined with one or more of the preceding paragraphs, the aqueous packaging solution for use in the packaging systems disclosed herein can further contain ectoine or an ophthalmologically acceptable ectoine derivative, e.g., hydroxyectoine. Ectoine is a natural substance which is obtained from microorganisms which live in extreme environments (e.g., salt lakes). These microorganisms form the natural substance ectoine in order to protect themselves from the extreme environmental factors prevailing there.

In an illustrative embodiment, the ectoine is L-ectoine ((S)-2-methyl-1,4,5,6-tetrahydropyrimidine-4-carboxylic acid). In an illustrative embodiment, ophthalmologically acceptable ectoine derivatives include, for example, hydroxyectoine ((4S,5S)-5-hydroxy-2-methyl-1,4,5,6-tetrahydropyrimidine-4-carboxylic acid), polyectoine, oliogomers thereof, salts, e.g. sodium- or potassium salts, of ectoine, esters which can be obtained by conversion of the 4-carboxy group with alcohols, in particular straight-chain or branched mono- or bivalent alcohols with 1 to 20 carbon atoms, and/or of the 5-hydroxy group with carboxylic acids, in particular straight-chain or branched-chain mono- or bivalent alkyl carboxylic acids with 2 to 20 carbon atoms, e.g., alkylmonocarboxylic acids, and also acid addition salts with inorganic or organic acids.

In accordance with one or more additional non-limiting illustrative embodiments, as may be combined with one or more of the preceding paragraphs, the ectoine or ophthalmologically acceptable ectoine derivative is present in the aqueous packaging solution in an amount ranging from about 0.01 to about 10 weight percent, based on the total weight of the aqueous packaging solution. In another illustrative embodiment, the ectoine or an ophthalmologically acceptable ectoine derivative is present in the aqueous packaging solution in an amount ranging from about 0.1 to about 2 weight percent, based on the total weight of the aqueous packaging solution.

In accordance with one or more additional non-limiting illustrative embodiments, as may be combined with one or more of the preceding paragraphs, the aqueous packaging solution for use in the packaging systems disclosed herein can further contain one or more glycosaminoglycans (GAG) other than hyaluronic acid or a salt thereof as discussed above. The GAGs other than hyaluronic acid or a salt thereof having commonly understood structures include those discussed hereinabove such as, for example, chondroitin sulfate (e.g., chondroitin 4- and 6-sulfates), heparan, heparin sulfate, heparosan, dermatan, dermatan sulfate, keratan sulfate, and other disaccharides such as sucrose, lactulose, lactose, maltose, trehalose, cellobiose, mannobiose and chitobiose.

In accordance with one or more additional non-limiting illustrative embodiments, as may be combined with one or more of the preceding paragraphs, the one or more GAG's can be further derivatized in a similar manner as discussed above with respect to derivatized hyaluronic acid to form a modified GAG.

In an illustrative embodiment, as may be combined with one or more of the preceding paragraphs, the one or more GAGs or modified GAGs other than hyaluronic acid or a salt or a derivative thereof as discussed above can be present in the aqueous packaging solution in an amount ranging from about 0.01 to about 1 weight percent, based on the total weight of the aqueous packaging solution. In another illustrative embodiment, the one or more GAGs or modified GAGs as discussed above can be present in the aqueous packaging solution in an amount ranging from about 0.01 to about 0.1 weight percent, based on the total weight of the aqueous packaging solution.

In an illustrative embodiment, as may be combined with one or more of the preceding paragraphs, the aqueous packaging solution further contains one or more antimicrobial agents. Representative examples of antimicrobial agents for use herein include, but are not limited to, peroxides, polyquaterniums, purite, and the like. Suitable peroxides include, for example, hydrogen peroxide, benzoyl peroxide, t-butyl-hydroperoxide, p-menthane hydroperoxide and the like and mixtures thereof. Suitable polyquaterniums include, for example, polyquaternium-1, polyquaterium-10, Polymer JR, and the like.

In an illustrative embodiment, as may be combined with one or more of the preceding paragraphs, the one or more antimicrobial agents such as peroxides are present in the aqueous packaging solution in an amount ranging from about 0.5 ppm to about 20 ppm, based on the total weight of the aqueous packaging solution. In another illustrative embodiment, the one or more antimicrobial agents such as peroxides are present in the aqueous packaging solution in an amount ranging from about 1 ppm to about 10 ppm, based on the total weight of the aqueous packaging solution.

In an illustrative embodiment, as may be combined with one or more of the preceding paragraphs, the aqueous packaging solution further contains one or more vitamin B5 agents. Suitable vitamin B5 agents include, for example, pantothenic acid, analogs or derivatives thereof, calcium pantothenate, sodium pantothenate, or panthenol.

In an illustrative embodiment, as may be combined with one or more of the preceding paragraphs, the one or more vitamin B5 agents are present in the aqueous packaging solution in an amount ranging from about 0.1 to about 2 weight percent, based on the total weight of the aqueous packaging solution. In another illustrative embodiment, the one or more vitamin B5 agents are present in the aqueous packaging solution in an amount ranging from about 0.5 to about 1 weight percent, based on the total weight of the aqueous packaging solution.

The packaging solutions disclosed herein are physiologically compatible. Specifically, the solution must be “ophthalmically safe” for use with a lens such as a contact lens, meaning that a contact lens treated with the solution is generally suitable and safe for direct placement on the eye without rinsing, that is, the solution is safe and comfortable for daily contact with the eye via a contact lens that has been wetted with the solution. An ophthalmically safe solution has a tonicity and pH that is compatible with the eye and includes materials, and amounts thereof, that are non-cytotoxic according to ISO standards and U.S. Food & Drug Administration (FDA) regulations.

The packaging solution should also be sterile in that the absence of microbial contaminants in the product prior to release must be statistically demonstrated to the degree necessary for such products. The liquid media useful in the present invention are selected to have no substantial detrimental effect on the lens being treated or cared for and to allow or even facilitate the present lens treatment or treatments. In an embodiment, the liquid media is aqueous-based. A particularly useful aqueous liquid medium is that derived from saline, for example, a conventional saline solution or a conventional buffered saline solution.

The pH of the aqueous packaging solutions should be maintained within the range of about 6 to about 9, or from about 6.5 to about 7.8. Suitable buffers may be added to the aqueous packaging solutions, including, for example, a borate buffer, potassium citrate, citric acid, sodium bicarbonate, TRIS and various mixed phosphate buffers (including combinations of Na₂HPO₄, NaH₂PO₄ and KH₂PO₄) and mixtures thereof. In an illustrative embodiment, a suitable tris (hydroxymethyl) aminomethane (2-amino-2-(hydroxymethyl) propane-1,3-diol), (also known as tromethamine, and commonly referred to as tris, tris buffer, tris base or TRIZMA, TRIZMA buffer or TRIZMA base) or a salt thereof such as TRIZMA HCL. In an illustrative embodiment, a suitable buffer is a borate buffer, containing one or more of boric acid, sodium borate, potassium tetraborate, potassium metaborate or mixtures of the same. Combinations of buffers are contemplated herein.

Generally, buffers will be used in amounts ranging from about 0.005 to about 2.5 weight percent, based on the total weight of the aqueous packaging solution. In an embodiment, buffers will be used in amounts ranging from about 0.01 to about 1.5 weight percent, based on the total weight of the aqueous packaging solution.

Typically, the aqueous packaging solutions are also adjusted with tonicity agents, to approximate the osmotic pressure of normal lacrimal fluids which is equivalent to a 0.9 percent solution of sodium chloride or 2.5 percent of glycerol solution. The aqueous packaging solutions are made substantially isotonic with physiological saline used alone or in combination, otherwise if simply blended with sterile water and made hypotonic or made hypertonic the lenses will lose their desirable optical parameters. Correspondingly, excess saline may result in the formation of a hypertonic solution which will cause stinging and eye irritation.

Suitable tonicity adjusting agents include, for example, sodium and potassium chloride, dextrose, calcium and magnesium chloride, mannitol, inositol, sorbitol, polyethylene glycol (PEG), glucose (dextrose), glycerin, glutamic acid, proline, stachydrine (which may also be known as N,N-dimethylproline or proline betaine), betaine (which may also be known as trimethylglycine or glycine betaine), taurine, L-carnitine, trehalose, gamma-aminobutyric acid (GABA), alanine, arginine, glycine, glutamine, asparagine, ornithine, isoleucine, leucine, valine, putrescine, spermidine, spermine, homospermine, cadaverine, urea, glycerophosphocholine and the like and mixtures thereof.

These tonicity adjusting agents are typically used individually in amounts ranging from about 0.01 to about 2.5% w/v. In an embodiment, the tonicity adjusting agents are used in amounts ranging from about 0.2 to about 1.5% w/v. The tonicity agent will be employed in an amount to provide a final effective osmotic value of at least about 150 mOsm/kg. In an embodiment, the tonicity adjusting agents are used in an amount to provide a final effective osmotic value of from about 150 to about 400 mOsm/kg. In an embodiment, the tonicity adjusting agents are used in an amount to provide a final effective osmotic value of from about 150 to about 350 mOsm/kg. In an embodiment, the tonicity adjusting agents are used in an amount to provide a final effective osmotic value of from about 160 to about 220 mOsm/kg.

If desired, one or more additional components can be included in the packaging solution. Such additional components are chosen to impart or provide at least one beneficial or desired property to the aqueous packaging solution. In general, the additional components may be selected from components which are conventionally used in one or more ophthalmic device care compositions. Suitable additional components include, for example, comfort agents, cleaning agents, wetting agents, nutrient agents, sequestering agents, viscosity builders, contact lens conditioning agents, antioxidants, and the like and mixtures thereof. These additional components may each be included in the packaging solutions in an amount effective to impart or provide the beneficial or desired property to the packaging solutions. For example, such additional components may be included in the packaging solutions in amounts similar to the amounts of such components used in other, e.g., conventional, contact lens care products.

In an illustrative embodiment, the aqueous packaging solution will further contain one or more comfort agents to provide additional lubricating and moisturizing characteristics to the aqueous packaging solutions. In an embodiment, a suitable comfort agent is one or more poloxamer comfort agents. A representative example of a suitable poloxamer comfort agent is a poloxamer block copolymer. One specific class of poloxamer block copolymers are those available under the trademark Pluronic (BASF Wyandotte Corp., Wyandotte, Mich.). Poloxamers include Pluronics and reverse Pluronics. Pluronics are a series of ABA block copolymers composed of poly (ethylene oxide)-poly (propylene oxide)-poly (ethylene oxide) blocks as generally represented in Formula (XXVI):

HO(C₂H₄O)ₐ(C₃H₆O)_{b}(C₂H₄O)ₐH (XXVI)

wherein a is independently at least 1 and b is at least 1. Another representative example of a poloxamer comfort agent is a poloxamer ABA block copolymer composed of poly (ethylene oxide)-poly (butylene oxide)-poly (ethylene oxide) blocks as generally represented in Formula XXVII:

HO(C₂H₄O)ₐ(C₄H₄O)_{b}(C₂H₄O)ₐH (XXVII)

Reverse Pluronics are a series of BAB block copolymers, respectively composed of poly (propylene oxide)-poly (ethylene oxide)-poly (propylene oxide) blocks as generally represented in Formula (XXVIII):

HO(C₃H₆O)_{b}(C₂H₄O)ₐ(C₃H₆O)_{b}H (XXVIII)

wherein a is at least 1 and b is independently at least 1.

Another representative example of a poloxamer comfort agent is a poloxamer BAB block copolymer composed of poly (butylene oxide)-poly (ethylene oxide)-poly (butylene oxide) blocks as generally represented in Formula XXIX:

HO(C₄H₈O)_{b}(C₂H₄O)ₐ(C₄H₈O)_{b}H (XXIX).

The poly (ethylene oxide), PEO, blocks are hydrophilic, whereas the poly (propylene oxide), PPO, and poly (butylene oxide), PBO, blocks are hydrophobic in nature. The poloxamers in each series have varying ratios of PEO and PPO or PBO which ultimately determines the hydrophilic-lipophilic balance (HLB) of the material, i.e., the varying HLB values are based upon the varying values of a and b, a representing the number of hydrophilic poly (ethylene oxide) units (PEO) being present in the molecule and b representing the number of hydrophobic poly (propylene oxide) units (PPO) or hydrophobic poly (butylene oxide) units (PBO) being present in the molecule. In an embodiment, the poloxamer will have an HLB ranging from about 5 to about 24. In an embodiment, the poloxamer will have an HLB ranging from about 1 to about 5.

Poloxamers and reverse poloxamers have terminal hydroxyl groups that can be terminal functionalized. An example of a terminal functionalized poloxamer and as discussed herein is poloxamer dimethacrylate (e.g., Pluronic® F127 dimethacrylate) as disclosed in U.S. Patent Application Publication No. 2003/0044468 and U.S. Pat. No. 9,309,357. Other examples include glycidyl-terminated copolymers of polyethylene glycol and polypropylene glycol as disclosed in U.S. Pat. No. 6,517,933.

The poloxamer is functionalized to provide the desired reactivity at the end terminal of the molecule. The functionality can be varied and is determined based upon the intended use of the functionalized PEO- and PPO- or PBO-containing block copolymers. That is, the PEO- and PPO- or PBO-containing block copolymers are reacted to provide end terminal functionality that is complementary with the intended device forming monomeric mixture. The term block copolymer as used herein shall be understood to mean a poloxamer as having two or more blocks in their polymeric backbone(s).

In an embodiment, a suitable comfort agent is one or more polyol comfort agents. Suitable polyols for use herein have the formula R″ (OH) y where R″ is a hydrocarbon radical and y is an integer representing the number of hydroxy radicals and has a value of from 2 to 6. The polyols may contain less than about 12 carbon atoms. Representative examples of polyol comfort agents include glycerol, propylene glycol, erythritol, and the like.

In an embodiment, a suitable comfort agent is one or more poloxamine comfort agents. While the poloxamers and reverse poloxamers are considered to be difunctional molecules (based on the terminal hydroxyl groups), the poloxamines are in a tetrafunctional form, i.e., the molecules are tetrafunctional block copolymers terminating in primary hydroxyl groups and linked by a central diamine. One specific class of poloxamine block copolymers are those available under the trademark Tetronic (BASF). Poloxamines include Tetronic and reverse Tetronics. Poloxamines have the following general structure of Formula (XXX):

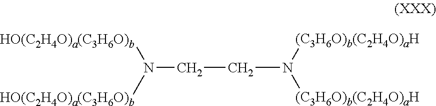

wherein a is independently at least 1 and b is independently at least 1.

The poloxamine is functionalized to provide the desired reactivity at the end terminal of the molecule. The functionality can be varied and is determined based upon the intended use of the functionalized PEO- and PPO-containing block copolymers. That is, the PEO- and PPO-containing block copolymers are reacted to provide end terminal functionality that is complementary with the intended device forming monomeric mixture. The term block copolymer as used herein shall be understood to mean a poloxamine as having two or more blocks in their polymeric backbone(s).

In an embodiment, a suitable comfort agent includes water soluble natural gums, cellulose-derived polymers and the like. Useful natural gums include guar gum, gum tragacanth and the like. Suitable cellulose-derived comfort components include, for example, cellulose-derived polymers, such as hydroxypropyl cellulose, hydroxypropylmethyl cellulose, carboxymethyl cellulose, methyl cellulose, hydroxyethyl cellulose and the like.

In an embodiment, a suitable comfort agent can include, for example, polyacrylic acid (PAA)-containing comfort agents such as, for example, PAA-g-amine, PAA-g-lactose, PAA-co-amine and the like. Another suitable comfort agent is PDMA-co-mPEG.

Combinations of any of the foregoing comfort agents are contemplated herein.

In general, the one or more comfort agents are present in the aqueous packaging solution in an amount ranging from about 0.01 to about 5 weight percent, based on the total weight of the aqueous packaging solution. In an illustrative embodiment, the one or more comfort agents are present in the aqueous packaging solution in an amount ranging from about 0.1 to about 1 weight percent, based on the total weight of the aqueous packaging solution.

Suitable sequestering agents include, for example, disodium ethylene diamine tetraacetate, alkali metal hexametaphosphate, citric acid, sodium citrate and the like and mixtures thereof.

Suitable viscosity builders include, for example, hydroxyethyl cellulose, hydroxymethyl cellulose, polyvinyl pyrrolidone, polyvinyl alcohol and the like and mixtures thereof.

Suitable antioxidants include, for example, sodium metabisulfite, sodium thiosulfate, N-acetylcysteine, butylated hydroxyanisole, butylated hydroxytoluene and the like and mixtures thereof.

The method of packaging and storing an ophthalmic device such as a contact lens according to illustrative embodiments includes at least packaging an ophthalmic device immersed in the aqueous packaging solution disclosed herein. The method may include immersing the ophthalmic device in an aqueous packaging solution prior to delivery to the customer/wearer, directly following manufacture of the contact lens. Alternately, the packaging and storing in the packaging solution may occur at an intermediate point before delivery to the ultimate customer (wearer) but following manufacture and transportation of the lens in a dry state, wherein the dry lens is hydrated by immersing the lens in the packaging solution. Consequently, a package for delivery to a customer may include a sealed container containing one or more unused contact lenses immersed in an aqueous packaging solution according to the embodiments described herein.

In an illustrative embodiment, the steps leading to the packaging system disclosed herein include (1) molding an ophthalmic device in a mold comprising at least a first and second mold portion, (2) hydrating and cleaning the ophthalmic device in a container optionally comprising at least one of the mold portions, (3) introducing the aqueous packaging solution disclosed herein into the container with the ophthalmic device supported therein, and (4) sealing the container. In an illustrative embodiment, the method further includes the step of sterilizing the contents of the container. Sterilization may take place prior to, or most conveniently after, sealing of the container and may be affected by any suitable method known in the art, e.g., by autoclaving of the sealed container at temperatures of about 120° C. or higher.

The following examples are provided to enable one skilled in the art to practice the invention and are merely illustrative of the invention. The examples should not be read as limiting the scope of the invention as defined in the claims.

### EXAMPLES 1 AND 2 AND COMPARATIVE EXAMPLES A AND B

An aqueous packaging solution was made by mixing the following components, listed in Table 1 at amounts per weight.

TABLE 1

| Ingredient | Comp. Ex. A | Comp. Ex. B | Ex. 1 | Ex. 2 |
|---|---|---|---|---|
| Sodium Phosphate | 0.027 | 0.027 | 0.027 | 0.027 |
| Monobasic | | | | |
| Monohydrate | | | | |
| Sodium Phosphate | 0.078 | 0.078 | 0.078 | 0.078 |
| Dibasic Anhydrous | | | | |
| Potassium Chloride | 0.832 | 0.832 | 0.832 | 0.832 |
| Poloxamine 1107 | 0.550 | 0.55 | 0.55 | 0.55 |
| Sodium Hyaluronate | 0.020 | 0.020 | 0.020 | 0.020 |
| Ectoine | 1.000 | 1.000 | 1.000 | 1.000 |
| Tremella | — | — | 0.010 | 0.010 |
| Hydrogen Peroxide | — | 0.033 | — | 0.033 |
| Sodium D- | 0.200 | 0.200 | 0.200 | 0.200 |
| Pantothenate (B5) | | | | |
| Purified Water, USP | 97.293 | 97.260 | 97.283 | 97.250 |

The aqueous packaging solutions of Examples 1 and 2 and Comparative Examples A and B were then subjected to a first autoclaving procedure at 121° C. and a second autoclaving procedure at 121° C. Solutions were mixed and placed in sealed glass vials before steam sterilization in the autoclave. The results of the pre- and post-autoclaving procedures are listed in Table 2.

TABLE 2

| Ingredient | Comp. Ex. A | Comp. Ex. B | Ex. 1 | Ex. 2 |
|---|---|---|---|---|
| pH (pre-autoclave) | 7.25 | 7.23 | 7.25 | 7.23 |
| pH (1X autoclave) | 7.32 | 7.33 | 7.30 | 7.29 |
| pH (2X autoclave) | 7.33 | 7.34 | 7.33 | 7.32 |
| OSMO, | 305 | 306 | 324 | 324 |
| (pre- autoclave) | | | | |
| OSMO, | 306 | 306 | 325 | 324 |
| (1X autoclave) | | | | |
| OSMO, | 307 | 307 | 325 | 325 |
| (2X autoclave) | | | | |
| Viscosity cP, | 1.758 | 1.804 | 1.958 | 1.722 |
| (pre- autoclave) | | | | |
| Viscosity cP, | 1.408 | 1.291 | 1.520 | 1.277 |
| (1X autoclave) | | | | |
| Viscosity cP, | 1.287 | 1.197 | 1.387 | 1.190 |
| (2X autoclave) | | | | |
| (MW) HA (kDa), | 2873 | 2804 | 2791 | 2620 |
| (pre- autoclave) | | | | |
| (MW) HA (kDa), | 1249 | 722 | 1559 | 964 |
| (1X autoclave) | | | | |
| (MW) HA (kDa), | 756 | 436 | 965 | 732 |
| (2X autoclave) | | | | |

### COMPARATIVE EXAMPLES C-E

An aqueous packaging solution was made by mixing the following components, listed in Table 3 at amounts per weight.

TABLE 3

| Ingredients | Comp Ex. C | Comp Ex. D | Comp Ex. E |
|---|---|---|---|
| Monobasic Sodium Phosphate | 0.015 | 0.015 | 0.015 |
| Dibasic Sodium Phosphate | 0.065 | 0.065 | 0.065 |
| NaCl | 0.9 | 0.9 | 0.9 |
| HA | 0.04 | 0.04 | 0.04 |
| Carnitine | — | 0.3 | — |
| Taurine | — | — | 0.3 |
| Water | 98.98 | 98.68 | 98.68 |
| TOTAL | 100 | 100 | 100 |
| pH | 7.21 | 7.23 | 7.09 |
| Osmo | 300 | 314 | 326 |

According to an aspect of the present disclosure, a packaging system for storage of an unused ophthalmic device comprises:
a sealed container containing one or more unused ophthalmic devices immersed in an aqueous packaging solution comprising hyaluronic acid or a salt or a derivative thereof and tremella; wherein the aqueous packaging solution has an osmolality of at least about 150 mOsm/kg, a pH of about 6 to about 9 and is sterilized.

In non-limiting illustrative embodiments, as may be combined with one or more of the preceding paragraphs, the hyaluronic acid or a salt or a derivative thereof has a weight average molecular weight of about 10,000 Daltons (Da) to about 4,000,000 Da.

In non-limiting illustrative embodiments, as may be combined with one or more of the preceding paragraphs, the hyaluronic acid or a salt or a derivative thereof has a weight average molecular weight of about 1,000,000 Da to about 3,200,000 Da.

In non-limiting illustrative embodiments, as may be combined with one or more of the preceding paragraphs, the hyaluronic acid or a salt or a derivative thereof has a weight average molecular weight of about 2,000,000 Da to about 2,800,000 Da.

In non-limiting illustrative embodiments, as may be combined with one or more of the preceding paragraphs, the hyaluronic acid or a salt or a derivative thereof is a hyaluronic acid derivative.

In non-limiting illustrative embodiments, as may be combined with one or more of the preceding paragraphs, the hyaluronic acid derivative comprises a reaction product of hyaluronic acid, a glycosaminoglycan other than hyaluronic acid or a salt thereof, and a first crosslinking agent.

In non-limiting illustrative embodiments, as may be combined with one or more of the preceding paragraphs, the glycosaminoglycan is chondroitin sulfate.

In non-limiting illustrative embodiments, as may be combined with one or more of the preceding paragraphs, one or more other glycosaminoglycans are crosslinked with the reaction product.

In non-limiting illustrative embodiments, as may be combined with one or more of the preceding paragraphs, the hyaluronic acid derivative comprises a glycophospholipid polymer comprising a reaction product of hyaluronic acid and one or more phospholipids.

In non-limiting illustrative embodiments, as may be combined with one or more of the preceding paragraphs, the one or more phospholipids comprise a phosphorylcholine-containing phospholipid.

In non-limiting illustrative embodiments, as may be combined with one or more of the preceding paragraphs, wherein the hyaluronic acid derivative comprises a grafted hyaluronic acid polymer comprising hyaluronic acid or a salt thereof having a polymer backbone and one or more side chains comprising a polyalkylene glycol-containing residue grafted onto the polymer backbone.

In non-limiting illustrative embodiments, as may be combined with one or more of the preceding paragraphs, the polyalkylene glycol-containing residue is derived from a polymeric compound or a salt thereof having the following structure:

Z—(((CH₂)ₐ—O)_{b})_{c}—Y

wherein Z is a reactive or a non-reactive end-capped group, Y is a reactive functional group, a is from 2 to 6, b is from 2 to 10,000 and c is 1 or 2.

In non-limiting illustrative embodiments, as may be combined with one or more of the preceding paragraphs, the hyaluronic acid derivative comprises a reaction product of (a) a grafted hyaluronic acid polymer comprising hyaluronic acid or a salt thereof having a polymer backbone and one or more side chains comprising a polyalkylene glycol-containing residue grafted onto the polymer backbone; and (b) one or more first crosslinking agents.

In non-limiting illustrative embodiments, as may be combined with one or more of the preceding paragraphs, the aqueous packaging solution comprises about 0.001 weight percent to about 5 weight percent, based on the total weight of the aqueous packaging solution, of the hyaluronic acid or a salt or a derivative thereof, and about 0.001 weight percent to about 5 weight percent, based on the total weight of the aqueous packaging solution, of tremella.

In non-limiting illustrative embodiments, as may be combined with one or more of the preceding paragraphs, the aqueous packaging solution comprises about 0.01 weight percent to about 1 weight percent, based on the total weight of the aqueous packaging solution, of the hyaluronic acid or a salt or a derivative thereof, and about 0.01 weight percent to about 1 weight percent, based on the total weight of the aqueous packaging solution, of tremella.

In non-limiting illustrative embodiments, as may be combined with one or more of the preceding paragraphs, the aqueous packaging solution further comprises ectoine or an ophthalmologically acceptable ectoine derivative.

In non-limiting illustrative embodiments, as may be combined with one or more of the preceding paragraphs, the ectoine is L-ectoine.

In non-limiting illustrative embodiments, as may be combined with one or more of the preceding paragraphs, the ophthalmologically acceptable ectoine derivative is hydroxyectoine, a polyectoine, a salt thereof, an ester thereof, an oliogomer thereof or a mixture thereof.

In non-limiting illustrative embodiments, as may be combined with one or more of the preceding paragraphs, the aqueous packaging solution comprises about 0.01 weight percent to about 10 weight percent, based on the total weight of the aqueous packaging solution, of the ectoine or the ophthalmologically acceptable ectoine derivative.

In non-limiting illustrative embodiments, as may be combined with one or more of the preceding paragraphs, the aqueous packaging solution further comprises a glycosaminoglycan selected from the group consisting of chondroitin, chondroitin sulfate, dermatan, dermatan sulfate, heparin, heparan sulfate, hyaluronan and mixtures thereof.

In non-limiting illustrative embodiments, as may be combined with one or more of the preceding paragraphs, the aqueous packaging solution comprises about 0.01 weight percent to about 0.05 weight percent, based on the total weight of the aqueous packaging solution, of the glycosaminoglycan or modified glycosaminoglycan.

In non-limiting illustrative embodiments, as may be combined with one or more of the preceding paragraphs, the aqueous packaging solution further comprises one or more antimicrobial agents.

In non-limiting illustrative embodiments, as may be combined with one or more of the preceding paragraphs, the one or more antimicrobial agents comprise one or more of a peroxide, polyquaternium, and purite.

In non-limiting illustrative embodiments, as may be combined with one or more of the preceding paragraphs, the peroxide includes one or more of hydrogen peroxide, benzoyl peroxide, t-butyl-hydroperoxide, and p-menthane hydroperoxide.

In non-limiting illustrative embodiments, as may be combined with one or more of the preceding paragraphs, the aqueous packaging solution comprises about 1 ppm to about 20 ppm, based on the total weight of the aqueous packaging solution, of the one or more antimicrobial agents.

In non-limiting illustrative embodiments, as may be combined with one or more of the preceding paragraphs, the aqueous packaging solution further comprises one or more vitamin B agents.

In non-limiting illustrative embodiments, as may be combined with one or more of the preceding paragraphs, the one or more vitamin B agents comprise sodium D-Pantothenate.

In non-limiting illustrative embodiments, as may be combined with one or more of the preceding paragraphs, the aqueous packaging solution comprises about 0.01 weight percent to about 2 weight percent, based on the total weight of the aqueous packaging solution, of the one or more vitamin B agents.

In non-limiting illustrative embodiments, as may be combined with one or more of the preceding paragraphs, the aqueous packaging solution further comprises one or more comfort agents.

In non-limiting illustrative embodiments, as may be combined with one or more of the preceding paragraphs, the aqueous packaging solution further comprises at least one of a poloxamer and a poloxamine.

In non-limiting illustrative embodiments, as may be combined with one or more of the preceding paragraphs, the packaging system further comprises one or more buffers.

In non-limiting illustrative embodiments, as may be combined with one or more of the preceding paragraphs, the aqueous packaging solution does not contain one or more of an effective disinfecting amount of a disinfecting agent and a germicide compound.

In non-limiting illustrative embodiments, as may be combined with one or more of the preceding paragraphs, the packaging system is heat sterilized two times subsequent to sealing of the package system.

In non-limiting illustrative embodiments, as may be combined with one or more of the preceding paragraphs, the packaging system is heat sterilized by autoclaving the package system.

According to another aspect of the present disclosure, a method of preparing a packaging system comprising a storable, sterile ophthalmic device comprises:
(a) providing an unused ophthalmic device;
(b) immersing the unused ophthalmic device in an aqueous packaging solution comprising (i) hyaluronic acid or a salt or a derivative thereof and (ii) tremella, wherein the aqueous packaging solution has an osmolality of at least about 150 mOsm/kg and a pH in the range of about 6 to about 9;
(c) packaging the aqueous packaging solution and the unused ophthalmic device in a manner preventing contamination of the unused ophthalmic device by microorganisms; and
(d) sterilizing the packaged solution and the unused ophthalmic device.

In non-limiting illustrative embodiments, as may be combined with one or more of the preceding paragraphs, the hyaluronic acid or a salt or a derivative thereof has a weight average molecular weight of about 10,000 Daltons (Da) to about 4,000,000 Da.

In non-limiting illustrative embodiments, as may be combined with one or more of the preceding paragraphs, the hyaluronic acid or a salt or a derivative thereof has a weight average molecular weight of about 1,000,000 Da to about 3,200,000 Da.

In non-limiting illustrative embodiments, as may be combined with one or more of the preceding paragraphs, the hyaluronic acid or a salt or a derivative thereof has a weight average molecular weight of about 2,000,000 Da to about 2,800,000 Da.

In non-limiting illustrative embodiments, as may be combined with one or more of the preceding paragraphs, the hyaluronic acid or a salt or a derivative thereof is a hyaluronic acid derivative.

In non-limiting illustrative embodiments, as may be combined with one or more of the preceding paragraphs, the hyaluronic acid derivative comprises a reaction product of hyaluronic acid, a glycosaminoglycan other than hyaluronic acid or a salt thereof, and a first crosslinking agent.

In non-limiting illustrative embodiments, as may be combined with one or more of the preceding paragraphs, the glycosaminoglycan is chondroitin sulfate.

In non-limiting illustrative embodiments, as may be combined with one or more of the preceding paragraphs, one or more other glycosaminoglycans are crosslinked with the reaction product.

In non-limiting illustrative embodiments, as may be combined with one or more of the preceding paragraphs, the hyaluronic acid derivative comprises a glycophospholipid polymer comprising a reaction product of hyaluronic acid and one or more phospholipids.

In non-limiting illustrative embodiments, as may be combined with one or more of the preceding paragraphs, the one or more phospholipids comprise a phosphorylcholine-containing phospholipid.

In non-limiting illustrative embodiments, as may be combined with one or more of the preceding paragraphs, the hyaluronic acid derivative comprises a grafted hyaluronic acid polymer comprising hyaluronic acid or a salt thereof having a polymer backbone and one or more side chains comprising a polyalkylene glycol-containing residue grafted onto the polymer backbone.

In non-limiting illustrative embodiments, as may be combined with one or more of the preceding paragraphs, the polyalkylene glycol-containing residue is derived from a polymeric compound or a salt thereof having the following structure:

Z—(((CH₂)ₐ—O)_{b})_{c}—Y

wherein Z is a reactive or a non-reactive end-capped group, Y is a reactive functional group, a is from 2 to 6, b is from 2 to 10,000 and c is 1 or 2.

In non-limiting illustrative embodiments, as may be combined with one or more of the preceding paragraphs, the hyaluronic acid derivative comprises a reaction product of (a) a grafted hyaluronic acid polymer comprising hyaluronic acid or a salt thereof having a polymer backbone and one or more side chains comprising a polyalkylene glycol-containing residue grafted onto the polymer backbone; and (b) one or more first crosslinking agents.

In non-limiting illustrative embodiments, as may be combined with one or more of the preceding paragraphs, the aqueous packaging solution comprises about 0.001 weight percent to about 5 weight percent, based on the total weight of the aqueous packaging solution, of the hyaluronic acid or a salt or a derivative thereof, and about 0.001 weight percent to about 5 weight percent, based on the total weight of the aqueous packaging solution, of tremella.

In non-limiting illustrative embodiments, as may be combined with one or more of the preceding paragraphs, the aqueous packaging solution comprises about 0.1 weight percent to about 1 weight percent, based on the total weight of the aqueous packaging solution, of the hyaluronic acid or a salt or a derivative thereof, and about 0.1 weight percent to about 1 weight percent, based on the total weight of the aqueous packaging solution, of tremella.

In non-limiting illustrative embodiments, as may be combined with one or more of the preceding paragraphs, the aqueous packaging solution further comprises ectoine or an ophthalmologically acceptable ectoine derivative.

In non-limiting illustrative embodiments, as may be combined with one or more of the preceding paragraphs, the ectoine is L-ectoine.

In non-limiting illustrative embodiments, as may be combined with one or more of the preceding paragraphs, the ophthalmologically acceptable ectoine derivative is hydroxyectoine, a polyectoine, a salt thereof, an ester thereof, an oliogomer thereof or a mixture thereof.

In non-limiting illustrative embodiments, as may be combined with one or more of the preceding paragraphs, the aqueous packaging solution comprises about 0.01 weight percent to about 10 weight percent, based on the total weight of the aqueous packaging solution, of the ectoine or the ophthalmologically acceptable ectoine derivative.

In non-limiting illustrative embodiments, as may be combined with one or more of the preceding paragraphs, the aqueous packaging solution further comprises a glycosaminoglycan selected from the group consisting of chondroitin, chondroitin sulfate, dermatan, dermatan sulfate, heparin, heparan sulfate, hyaluronan and mixtures thereof.

In non-limiting illustrative embodiments, as may be combined with one or more of the preceding paragraphs, the aqueous packaging solution comprises about 0.01 weight percent to about 0.05 weight percent, based on the total weight of the aqueous packaging solution, of the modified glycosaminoglycan.

In non-limiting illustrative embodiments, as may be combined with one or more of the preceding paragraphs, the aqueous packaging solution further comprises one or more antimicrobial agents.

In non-limiting illustrative embodiments, as may be combined with one or more of the preceding paragraphs, the one or more antimicrobial agents comprise one or more of a peroxide, polyquaternium, and purite.

In non-limiting illustrative embodiments, as may be combined with one or more of the preceding paragraphs, the peroxide includes one or more of hydrogen peroxide, benzoyl peroxide, t-butyl-hydroperoxide, and p-menthane hydroperoxide.

In non-limiting illustrative embodiments, as may be combined with one or more of the preceding paragraphs, the aqueous packaging solution comprises about 1 ppm to about 20 ppm, based on the total weight of the aqueous packaging solution, of the one or more antimicrobial agents.

In non-limiting illustrative embodiments, as may be combined with one or more of the preceding paragraphs, the aqueous packaging solution further comprises one or more vitamin B agents.

In non-limiting illustrative embodiments, as may be combined with one or more of the preceding paragraphs, the one or more vitamin B agents comprise sodium D-Pantothenate.

In non-limiting illustrative embodiments, as may be combined with one or more of the preceding paragraphs, the aqueous packaging solution comprises about 0.01 weight percent to about 2 weight percent, based on the total weight of the aqueous packaging solution, of the one or more vitamin B agents.

In non-limiting illustrative embodiments, as may be combined with one or more of the preceding paragraphs, the aqueous packaging solution further comprises one or more comfort agents.

In non-limiting illustrative embodiments, as may be combined with one or more of the preceding paragraphs, the aqueous packaging solution further comprises at least one of a poloxamer and a poloxamine.

In non-limiting illustrative embodiments, as may be combined with one or more of the preceding paragraphs, the aqueous packaging solution further comprises one or more buffers.

In non-limiting illustrative embodiments, as may be combined with one or more of the preceding paragraphs, the aqueous packaging solution does not contain one or more of an effective disinfecting amount of a disinfecting agent and a germicide compound.

In non-limiting illustrative embodiments, as may be combined with one or more of the preceding paragraphs, the sterilizing the packaged solution and the ophthalmic device comprises heat sterilizing the packaged solution and the ophthalmic device two times subsequent to sealing of the package system.

In non-limiting illustrative embodiments, as may be combined with one or more of the preceding paragraphs, the sterilizing the packaged solution and the ophthalmic device comprises autoclaving the package system.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present formulations and are disclosed herein just as if each and every such sub-combination was individually and explicitly disclosed herein.

It will be understood that various modifications may be made to the embodiments disclosed herein. Therefore, the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A packaging system for storage of an unused ophthalmic device comprising:
a sealed container containing one or more unused ophthalmic devices immersed in an aqueous packaging solution comprising hyaluronic acid or a salt or a derivative thereof and tremella; wherein the aqueous packaging solution has an osmolality of at least about 150 mOsm/kg, a pH of about 6 to about 9 and is sterilized.

2. The packaging system according to claim 1, wherein the hyaluronic acid derivative comprises a reaction product of hyaluronic acid, a glycosaminoglycan other than hyaluronic acid or a salt thereof, and a first crosslinking agent.

3. The packaging system according to claim 1, wherein the hyaluronic acid derivative comprises a glycophospholipid polymer comprising a reaction product of hyaluronic acid and one or more phospholipids.

4. The packaging system according to claim 3, wherein the one or more phospholipids comprise a phosphorylcholine-containing phospholipid.

5. The packaging system according to claim 1, wherein the hyaluronic acid derivative comprises a grafted hyaluronic acid polymer comprising hyaluronic acid or a salt thereof having a polymer backbone and one or more side chains comprising a polyalkylene glycol-containing residue grafted onto the polymer backbone.

6. The packaging system according to claim 5, wherein the polyalkylene glycol-containing residue is derived from a polymeric compound or a salt thereof having the following structure:
Z—(((CH₂)ₐ—O)_{b})_{c}—Y
wherein Z is a reactive or a non-reactive end-capped group, Y is a reactive functional group, a is from 2 to 6, b is from 2 to 10,000 and c is 1 or 2.

7. The packaging system according to claim 1, wherein the hyaluronic acid derivative comprises a reaction product of (a) a grafted hyaluronic acid polymer comprising hyaluronic acid or a salt thereof having a polymer backbone and one or more side chains comprising a polyalkylene glycol-containing residue grafted onto the polymer backbone; and (b) one or more first crosslinking agents.

8. The packaging system according to claim 1, wherein the aqueous packaging solution comprises about 0.001 weight percent to about 5 weight percent, based on the total weight of the aqueous packaging solution, of the hyaluronic acid or a salt or a derivative thereof, and about 0.001 weight percent to about 5 weight percent, based on the total weight of the aqueous packaging solution, of tremella.

9. The packaging system according to claim 1, wherein aqueous packaging solution further comprises ectoine or an ophthalmologically acceptable ectoine derivative.

10. The packaging system according to claim 9, wherein the aqueous packaging solution comprises about 0.01 weight percent to about 10 weight percent, based on the total weight of the aqueous packaging solution, of the ectoine or the ophthalmologically acceptable ectoine derivative.

11. The packaging system according to claim 1, wherein the aqueous packaging solution further comprises one or more antimicrobial agents.

12. The packaging system according to claim 11, wherein the one or more antimicrobial agents comprise one or more of a peroxide, polyquaternium, and purite.

13. The packaging system according to claim 12, wherein the peroxide includes one or more of hydrogen peroxide, benzoyl peroxide, t-butyl-hydroperoxide, and p-menthane hydroperoxide.

14. The packaging system according to claim 11, wherein the aqueous packaging solution comprises about 1 ppm to about 20 ppm, based on the total weight of the aqueous packaging solution, of the one or more antimicrobial agents.

15. The packaging system according to claim 1, wherein the aqueous packaging solution further comprises one or more vitamin B agents.

16. The packaging system according to claim 15, wherein the one or more vitamin B agents comprise sodium D-Pantothenate.

17. The packaging system according to claim 15, wherein the aqueous packaging solution comprises about 0.01 weight percent to about 2 weight percent, based on the total weight of the aqueous packaging solution, of the one or more vitamin B agents.

18. The packaging system according to claim 1, wherein the aqueous packaging solution further comprises one or more comfort agents.

19. The packaging system according to claim 1, wherein the packaging system is heat sterilized two times subsequent to sealing of the package system.

20. A method of preparing a packaging system comprising a storable, sterile ophthalmic device, the method comprising:
(a) providing an unused ophthalmic device;
(b) immersing the unused ophthalmic device in an aqueous packaging solution comprising (i) hyaluronic acid or a salt or a derivative thereof and (ii) tremella, wherein the aqueous packaging solution has an osmolality of at least about 150 mOsm/kg and a pH in the range of about 6 to about 9;
(c) packaging the aqueous packaging solution and the unused ophthalmic device in a manner preventing contamination of the unused ophthalmic device by microorganisms; and
(d) sterilizing the packaged solution and the unused ophthalmic device.
